SEQUENCE LISTING (8 sequences)

SEQ ID NO: 1 - DNA, 32 bp, Artificial
  Forward primer for amplifying Myc-tagged MCU
ataagcttatggcggccgccgcaggtagatcg

SEQ ID NO: 2 - DNA, 57 bp, Artificial
  Reverse primer for amplifying Myc-tagged MCU
ctacaggtcttcttcgctaatcagtttctgttcatctttttcaccaatttgtcggag

SEQ ID NO: 3 - DNA, 36 bp, Artificial
  Forward primer for amplifying pAd5CMVmcsIRESeGFP
gaagaagacctgtaggatatcgaattcctgcagccc

SEQ ID NO: 4 - DNA, 32 bp, Artificial
  Reverse primer for amplifying pAd5CMVmcsIRESeGFP
gccgccataagcttatcgataccgtcgacctc

SEQ ID NO: 5 - DNA, 32 bp, Artificial
  Forward primer for amplifying DN-MCU DNA product
accaacgtcgacatggcggccgccgcaggtag

SEQ ID NO: 6 - DNA, 70 bp, Artificial
  Reverse primer for amplifying DN-MCU DNA product
gaacgcaagcttcctacaggtcttcttcgctaatcagtttctgttcatctttttcaccaatttgtcggag

SEQ ID NO: 7 - DNA, 25 bp, Artificial
  Forward primer for amplifying DN-MCU
cccacaccagaaatgacagacagat

SEQ ID NO: 8 - DNA, 23 bp, Artificial
  Reverse primer for amplifying DN-MCU
agaggagcagcaggagcgatcta

(19) United States
(12) Patent Application Publication
(10) Pub. No.: US 2016/0095837 A1
(43) Pub. Date: Apr. 7, 2016

(54) MODULATION OF MITOCHONDRIAL CALCIUM UNIPORTER ACTIVITY FOR TREATING AND PREVENTING ARRHYTHMIAS

(71) Applicant: University of Iowa Research Foundation, Iowa City, IA (US)

(72) Inventors: Mark E. Anderson, Iowa City, IA (US); Yuejin Wu, Iowa City, IA (US)

(73) Assignee: UNIVERSITY OF IOWA RESEARCH FOUNDATION, Iowa City, IA (US)

(21) Appl. No.: 14/877,433

(22) Filed: Oct. 7, 2015

Related U.S. Application Data
(60) Provisional application No. 62/060,783, filed on Oct. 7, 2014.

(51) Int. Cl. A61K 31/28; C07K 14/705

(52) U.S. Cl. CPC A61K 31/28; C07K 14/705

(57) ABSTRACT

Disclosed are methods and compositions for treating or preventing diseases or disorders associated with mitochondrial calcium uniporter (MCU) activity. The methods may include administering a modulator of MCU activity to a patient in need thereof.

### CROSS-REFERENCE TO RELATED PATENT APPLICATIONS

The present application claims the benefit of priority under 35 U.S.C. §119(e) to U.S. Provisional Patent Application No. 62/060,783, filed on Oct. 7, 2014, the content of which is incorporated by reference in its entirety.

### STATEMENT REGARDING GOVERNMENT SPONSORED RESEARCH OR DEVELOPMENT

This invention was made with government support under Grant Nos. R01-HL079031, R01-HL070250, and R01-HL071140 awarded by the National Institutes of Health. The Government has certain rights in this invention.

### BACKGROUND

The field of the invention relates to treatment and prevention of diseases and disorders associated with mitochondrial calcium uniporter (MCU). In particular the field of the invention relates to modulating mitochondrial calcium uniporter activity for treating and preventing heart diseases and disorders such as arrhythmias.

Heart rhythm problems (i.e., arrhythmias) occur when the electrical impulses in a heart that coordinate heartbeats do not work properly, causing the heart to beat too fast, too slow or irregularly. Heart arrhythmias may feel like a fluttering or racing heart, and are often harmless. However, some heart arrhythmias may cause bothersome, sometimes even life-threatening, signs and symptoms. Heart arrhythmia treatment can often control or eliminate irregular heartbeats. Troublesome heart arrhythmias are often made worse, or are even caused, by a weak or damaged heart. Treatment is required only if the arrhythmia is causing significant symptoms or if the arrhythmia is putting a patient at risk of a more serious arrhythmia or arrhythmia complication.

Slow heartbeats (i.e., bradycardias) that do not have a cause that can be corrected, such as low thyroid hormone levels or a drug side effect, often are treated with a pacemaker. Fast heartbeats (i.e., tachycardias) may be treated in a number of ways.

For example, Vagal maneuvers may be used to treat supraventricular tachycardia (SVT) and include holding ones breath and straining, dunking ones face in ice water, or coughing. Medications may be administered, and while medications may not cure the problem, medications can reduce episodes of tachycardia or slow down the heart when an episode occurs. However, some medications may slow down a patient's heart so much that the patient requires a pacemaker. For atrial fibrillation, doctors will likely prescribe blood-thinning medication such as Coumadin (Warfarin®), dabigatran (Pradaxa®), or rivaroxaban (Xarelto®). Ablation therapy also may be administered. In ablation therapy, one or more catheters are threaded through blood vessels to the inner heart and positioned on tissues of the heart believed to be the sources of arrhythmia. Electrodes at the catheter tips then are heated to ablate the problem tissues, or alternatively, the catheter tips are cooled to freeze the problem tissue. Either method destroys a small spot of heart tissue and creates an electrical block along the electrophysiochemical pathway that is causing arrhythmia. Surgery also may be practiced to remove problem tissue.

About 14 million people in the USA have arrhythmias (i.e., ˜5% of the population). The most common disorders are atrial fibrillation and flutter. The incidence is highly related to age and the presence of underlying heart disease and approaches 30% following open heart surgery. There are many different types of antiarrhythmic drugs and many different mechanisms of action. Most of the drugs affect ion channels that are involved in the movement of sodium, calcium and potassium ions in and out of the cell. These drugs include mechanistic classes such as sodium-channel blockers, calcium-channel blockers and potassium-channel blockers. By altering the movement of these important ions, the electrical activity of the cardiac cells, including both pacemaker and non-pacemaker cells, is altered, hopefully in a manner that suppresses arrhythmias. Other drugs affect autonomic influences on the heart, which may stimulate or aggravate arrhythmias. Among these drugs are beta-blockers. However, current therapies to control heart rate acceleration often are ineffective and may lead to disabling side effects (e.g., asthma, depression, edema) and may slow basal heart rates. Excessive basal heart rate slowing can lead to loss of consciousness, heart failure, ventricular arrhythmias and may require surgical implantation of artificial pacemakers.

We have determined that the mitochondrial calcium uniporter (MCU) is an important factor in controlling heart rate acceleration. During intracellular Ca²⁺ signaling, mitochondria accumulate significant amounts of Ca²⁺ from the cytosol. Mitochondrial Ca²⁺ uptake controls the rate of energy production, shapes the amplitude and spatio-temporal patterns of intracellular Ca²⁺ signals, and is instrumental to cell death. This Ca²⁺ uptake is undertaken by the mitochondrial Ca²⁺ uniporter (MCU) located in the organelle's inner membrane. The uniporter passes Ca²⁺ down the electrochemical gradient maintained across this membrane without direct coupling to ATP hydrolysis or transport of other ions. (See Kirichok, “The mitochondrial calcium uniporter is a highly selective ion channel,” Nature 427, 360-364 (22 Jan. 2004)). Others have suggested methods for treating cardiac arrhythmia by administering gene therapy to modulate an electrical property of a pacemaker cell. (See U.S. Published Application No. 2007/0048484). However, to the best of the current inventors' knowledge, there are no known therapeutic treatments related to modulation of the MCU specifically.

Here, we disclose new mitochondrial-targeted reagents and a therapeutic strategy to treat or prevent heart diseases and disorders by modulating MCU activity. In particular, we have determined that MCU activity may be inhibited in order to control heart rate acceleration without slowing basal heart rate.

### SUMMARY

Disclosed are methods and composition for treating or preventing a disease or disorder associated with mitochondrial calcium channel (MCU) activity in a patient in need thereof. The methods may include administering a pharmaceutical composition comprising an agent that modulates MCU activity. Suitable diseases and disorders that are treated or prevented may include heart diseases and disorders, such as cardiac arrhythmia and in particular, atrial fibrillation.

The disclosed methods may be practiced in order to reduce heart rate acceleration in the patient, for example where the methods reduce heart rate acceleration in the patient by at least about 10%, 20%, 30%, 40%, 50%, or greater. Preferably, the disclosed methods do not reduce basal heart rate in the patient, for example where the methods do not reduce basal heart rate in the patient by more than 10%.

Suitable agents for modulating MCU activity may include MCU inhibitors such as MCU antagonist (e.g., Ru360). Other suitable MCU inhibitors may include polynucleotides encoding dominant negative mutants of MCU, for example where the polynucleotides are expressed from recombinant viral expression vectors.

In the disclosed methods, the agent for modulating MCU activity may be administered to the patient by any suitable route. Where the agent is a polynucleotide encoding a dominant negative mutant of MCU expressed from a recombinant viral expression vector, the polynucleotide may be administered via gene painting on cardiac tissue of the patient.

Also disclosed herein are pharmaceutical compositions comprising agents that modulate MCU activity. In some embodiments of the pharmaceutical compositions, the agent that modulated MCU activity is a polynucleotide encoding a dominant negative mutant of MCU expressed from a viral vector. Suitable viral vectors may include adenovirus vectors.

### BRIEF DESCRIPTION OF THE DRAWINGS

FIG. 1 SAN cell, ex-vivo and in-vivo heart rate response to ISO is impaired by Ru360 or DN-MCU, but rescued by ATP. a-c, Example action potential (AP) tracings recorded under basal (left) and ISO stimulated (right) conditions from freshly isolated SAN cells. The horizontal line marks 0 mV. Scale bars are 100 ms horizontal and 20 mV vertical. d, Summary dose-response data for SAN cell AP rate responses to ISO. e-g, Example AP tracings recorded under basal and ISO stimulated conditions from cultured SAN cells. The horizontal line marks 0 mV. Scale bars are the same as in (a-c). h, Summary dose-response data for cultured SAN cell AP rate responses to ISO. i-j, Example ex-vivo ECG tracings recorded under basal (left) and ISO stimulated (right) conditions from gene painted hearts. Scale bar is 100 ms. k, Summary dose-response data for ex-vivo heart rate response to ISO. l-m, Example in vivo ECG tracings recorded under basal (left) and ISO stimulated (right) conditions from DN-MCU and littermate WT mice. Scale bar is 100 ms. n, Summary data for in-vivo heart rate response to ISO (10 μg/kg). The solid bars representing baseline and hashed bars representing ISO response. *p<0.05, **p<0.01, ***p<0.001, n=10-15/group in panel d, n=5-9/group in panel h, n=22-24/group in panel k and n=5-6/group in panel n. Error bars indicate SEM.

FIG. 2 ISO induced acceleration of cytoplasmic Ca²⁺ sequestration is reduced by Ru360 but rescued by ATP. a-c. Example averaged tracings show baseline (black) and ISO-stimulated (blue) intracellular Ca²⁺ transients during spontaneous SAN cell activity measured under conditions as shown in FIG. 1a-c. Ca² transients were normalized to peak values for analysis of the decay phase. The decay phase of each tracing (marked by blue background), between the dashed lines marking 90-10% of peak values, shows rates of cytoplasmic Ca²⁺ sequestration. Scale bars are 100 ms. d, Summary data for the cytoplasmic Ca²⁺ decay slope from 90% to 10% of the peak amplitude before and after ISO. *p<0.05 versus all other groups. n=5-10/group. e, Superimposed (baseline) traces from panels a-c show Ru360 or Ru360+ATP did not affect baseline Ca²⁺ sequestration compared to control (−Ru360). Scale bar is 100 ms. f-i, The PLN super-inhibitor N27A mutant slowed SAN cell rates and cytoplasmic Ca²⁺ sequestration by a mechanism that was resistant to rescue by ATP dialysis. f and g, Example tracings as in (a-c). Scale bars are 100 ms. h, Summary data for the slope of the cytoplasmic Ca²⁺ decay, as in (d), NS between the groups. n=5-10/group. i, Summary dose-response data for AP rate responses to ISO in SAN cells from N27A transgenic mice with and without ATP dialysis. NS for all comparisons, n=5-10/group. Error bars indicate SEM.

FIG. 3 ISO increases SAN cell mitochondrial Ca²⁺. a, Adenovirus infected SAN cells expressing mito pericam co-localized with mito-tracker (scale bar=10 μm). b, The mito pericam fluorescence signal was increased by ISO (100 nM) stimulation and Ru360 dialysis prevented this increase (scale bar=10 μm). c, Example tracings show ISO increases in mitochondrial Ca²⁺ (Caₘᵢₜₒ) were prevented by Ru360 dialysis (scale bar=100 seconds). d, Summary data for Caₘᵢₜₒ responses to ISO. ***p<0.001, n=14-15/group. Error bars indicate SEM.

FIG. 4 Ru360 prevents ISO-stimulated increases in NADH. a, Example of NADH fluorescence responses to ISO (100 nM) in control (−Ru360) and Ru360 dialyzed SAN cells. Scale is 100 seconds. b, Summary data for NADH fluorescence responses to ISO. ***p<0.001, n=7-18/group. Error bars indicate SEM. c, Proposed mechanism for MCU-enhancing SAN responses to ISO. βAR: beta adrenergic receptor, CaV: Voltage dependent Ca²⁺ channels. HCN: Hyperpolarization-activated cyclic nucleotide-gated channels, MCU: Mitochondrial calcium uniporter, DN-MCU: dominate negative MCU, N27A: super-inhibitory phospholamban, NCX: Na⁺/Ca²⁺ Exchanger, PLN: Phospholamban, SERCA: Sarco/endoplasmic reticulum Ca²⁺-ATPase, SR: Sarcoplasmic reticulum, TG: thapsigargin.

FIG. 5. SAN AP rates are unaffected by Ru360 or ATP dialysis. a, The open bars represent stable AP rates after achieving whole cell mode access and hashed bars represent AP rates after ≧10 min dialysis with the pipette solution; NS at onset and after 10 min dialysis in each group and between the groups, n=4-15/group. b, ATP dialysis (8 mM) does not enhance SAN cell rate response to ISO. NS between groups under any condition, n=4-15/group. Error bars indicate SEM.

FIG. 6. Ru360 and DN-MCU expression prevent MCU Ca²⁺ uptake in permeabilized HEK cells. a-d. Representative tracings of 3 similar experiments in HEK cells. Baseline fluorescence was normalized to 1 and arrows indicate addition of 5 μM of CaCl₂. Ru360 was added as labeled (10 μM).

FIG. 7. Maximal action potential rate response to ISO in isolated SAN cells was reduced by MCU inhibition with Ru360 dialysis or DN-MCU expression and rescued by dialysis with ATP. n=5-15/group, *p<0.05, **p<0.01. Error bars indicate SEM.

FIG. 8. Immunofluorescent staining of (a) control left atrium and (b) gene painted SAN showing DAPI (blue) and HCN4 (red) to identify SAN, and myc epitope tag (green) for DN-MCU-myc expression. Scale bars are 200 μm.

FIG. 9. Schematic of the transgenic construct providing cardiomyocyte delimited expression of DN-MCU-myc from the alpha-MHC promoter. a, Restriction enzyme sites used for subcloning and linearization as described in the Methods are shown. hGH, human growth hormone poly-A signal. b, Western blots for myc tagged DN-MCU protein from heart (H), liver (L) and skeletal muscle (S) lysates (WT, n=3; DN-MCU, n=3). c, Summary echocardiographic data showing mean±standard deviation (WT, n=21; DN-MCU, n=20). No significant difference was identified in any parameter: resting heart rate (HR), ejection fraction (EF), left ventricular mass (LV mass), end diastolic volume (EDV), end systolic volume (ESV), stroke volume (SV), and cardiac output (CO). d-e, Representative Ca²⁺ uptake tracings of 3 similar experiments in permeabilized cardiac myocytes from DN-MCU and littermate WT mice (as in FIG. 6). Each arrow represents addition of 100 μM Ca²⁺.

FIG. 10. Ryanodine and Thapsigargin reduce SAN cell beating rate at baseline and prevent the ISO response and rescue by ATP dialysis (see FIG. 1 for comparison). a, Summary data for percent reduction of SAN cell action potential rates by ryanodine and thapsigargin. NS between the groups, n=5-6/group. b, Summary data for action potential rate responses to ISO in SAN cells treated with ryanodine, thapsigargin and ATP dialysis. NS between the groups, n=4-7/group. Error bars indicate SEM. c-d, phosphorylation of PLN at threonine 17 and serine16 were not significantly altered in atrial from DN-MCU mice at baseline. ISO treatment increased phosphorylation level of PLN at both serine 16 and threonine 17 sites similarly. Each bar represents averaged data from 7-9 mice. * p<0.05.

FIG. 11. I_{f} responses to ISO are MCU independent. a, Representative I_{f} current recordings in response to voltage clamp commands (top panel) from an SAN cell without Ru360 (middle panel) and an Ru360 dialyzed SAN cell (lower panel) before (left) and after ISO (right). b, Summary current-voltage relationship for I_{f} from groups shown in panel a; n=4 SAN cells without Ru360 (−Ru360) and n=5 Ru360-dialyzed cells (+Ru360). NS for I_{f} current density in Ru360-dialyzed compared to SAN cells without Ru360 before and after ISO. Error bars indicate SEM.

FIG. 12. I_{Ca} responses to ISO are MCU independent. a, Representative I_{Ca} current recordings in response to voltage clamp commands (top panel) from an SAN cell without Ru360 (−Ru360, middle panel) and an Ru360-dialyzed SAN cell (+Ru360, lower panel) before (black trace) and after ISO (blue trace). b, Summary current-voltage relationship for I_{Ca} from groups shown at left. n=6-8/group. NS for I_{Ca} current density in +Ru360 compared to −Ru360 cells before and after ISO. Error bars indicate SEM.

FIG. 13. Ru360 prevents ISO triggered increases in NADH. a, Example NADH fluorescence tracing in an SAN cell without Ru360 dialysis (−Ru360) shows responses to ISO, FCCP and rotenone. b, Example NADH fluorescence tracing in an Ru360-dialyzed SAN cell (+Ru360) where the response to ISO was abolished. We normalized NADH levels after FCCP as 0% and rotenone induced NADH change as 100%. Baseline level NADH was 22±3%, n=15/SEM.

### DETAILED DESCRIPTION

The disclosed subject matter further may be described utilizing terms as defined below.

Unless otherwise specified or indicated by context, the terms “a”, “an”, and “the” mean “one or more.” For example, “an inhibitor of MCU activity” should be interpreted to mean “one or more inhibitors of MCU activity.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≦10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the term “patient” may be used interchangeably with the term “subject” or “individual” and may include an “animal” and in particular a “mammal.” Mammalian subjects may include humans and other non-human primates, domestic animals, farm animals, and companion animals such as dogs, cats, guinea pigs, rabbits, rats, mice, horses, cattle, cows, and the like.

A “patient in need thereof” is intended to include a patient having or at risk for developing diseases and disorders associated with mitochondrial calcium uniporter (MCU) activity. In particular, a “patient in need thereof” is intended to include a patient having or at risk for developing heart diseases and disorders associated with MCU activity. Such diseases may include, but are not limited to arrhythmias, ischemic heart disease, congestive heart failure, heart attacks, strokes, and heart valve problems. In some embodiments, the disclosed pharmaceutical compositions are administered to treat or prevent arrhythmias, which may include but are not limited to atria fibrillation and tachycardias.

In order to treat or prevent a disease or disorder associated with MCU activity, a patient in need thereof may be administered a therapeutically effective amount of an agent that modulates MCU activity (e.g., an inhibitor or MCU activity). As used herein, the phrase “therapeutically effective amount” shall mean that drug dosage that provides the specific pharmacological response for which a therapeutic agent is administered in a significant number of subjects in need of such treatment. A therapeutically effective amount of a therapeutic agent that is administered to a particular subject in a particular instance will not always be effective in treating the conditions/diseases described herein, even though such dosage is deemed to be a therapeutically effective amount by those of skill in the art.

As used herein, the terms “treatment,” “treat,” or “treating” refer to therapy or prophylaxis of diseases, disorders, and the symptoms thereof in a subject in need thereof. Therapy or prophylaxis typically results in beneficial or desirable clinical effects, such as alleviation of symptoms, diminishment of extent of disease, stabilization (i.e., not worsening) of the state of the disease, delay or slowing of disease progression, amelioration or palliation of the disease state, and remission (whether partial or total and, whether detectable or undetectable). “Treatment” can also mean prolonging survival as compared to expected survival if a patient were not to receive treatment. Those in need of treatment include those already with the condition or disorder as well as those prone to have the condition or disorder or those in which the condition or disorder is to be prevented.

The methods disclosed herein may include methods of modulating MCU activity. As used herein, “modulating” means “changing” or “regulating” and may include “inhibiting” MCU activity. The methods may utilize an inhibitor of MCU activity which specifically inhibits MCU activity. A specific inhibitor of MCU preferably reduces MCU activity by at least 50% (or 60%, 70%, 80%, 90%, or 95%) when administered at a given concentration but does not reduce activity of a non-MCU calcium channel by more than 20% (or 15%, 10%, or 5%) when administered at the same concentration. Inhibitors of MCU activity may include channel blockers or non-channel blockers. Inhibitors of MCU activity may include Ru360 otherwise known as (μ)[(HCO₂)(NH₃)₄Ru]₂OCl₃), having a molecular formula (i.e., C₂H₂₆N₈O₅Ru₂.3Cl and a structure:

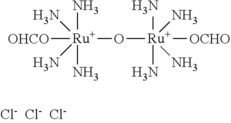

RU360 is cell-permeable oxygen-bridged dinuclear ruthenium amine complex that has been shown to bind to mitochondria with high affinity (K_{d}=340 pM). RU360 specifically blocks Ca²⁺ uptake into mitochondria in vitro (IC₅₀=184 pM) and in situ in intact myocytes and completely blocks Ca²⁺ uptake into mitochondria after incubation with ˜10 μM of Ru360 for 30 min. RU360 does not affect other cellular Ca²⁺ transport processes involved in cardiac muscle contraction, even at micromolar levels.

Inhibitors of MCU activity may include dominant negative mutants of MCU (e.g., DN-MCU or fragments thereof that bind to MCU and inhibit the activity of MCU). (See e.g., Raffaello et al., The mitochondrial calcium uniporter is a multimer that can include a dominant-negative pore-forming subunit,” EMBO J. 2013 August 28; 32(17):2362-76; and Lee et al., Structure and function of the N-terminal domain of the human mitochondrial calcium uniporter, EMBO Rep 2015 Ocxt; 16(10:1318-33; the contents of which are incorporate herein by reference in their entireties). For example, a patient in need thereof may be administered an expression vector (e.g., a recombinant viral expression vector), that expresses a dominant negative mutant of MCU (e.g., DN-MCU or fragments thereof that bind to MCU and inhibit the activity of MCU).

The disclosed methods may be used to decrease heart rate in a patient. A decrease in heart rate may be measured according to one or more of standard electrophysiological assays or standard electrocardiogram (ECG) recording. (See U.S. Published Application No. 2007/2007/0048484, which is incorporated herein by reference in its entirety.) In some embodiments, the disclosed methods may be used to decrease a patient's heart rate by at least about 10% relative to baseline in the electrophysiological assay, preferably by at least about 20%, 30%, 40%, 50% or more. In additional embodiments, the disclosed methods may be used to increase the atrio-ventricular (AV) node refractory period (AVNERP) as measured by the electrophysiological assay, by at least about 10% relative to baseline in the assay, preferably by at least about 20%, 30%, 40%, 50% or more. Conventional methods for detecting and measuring the AVNERP are known and include the standard electrophysiological tests. (See U.S. Published Application No. 2007/2007/0048484, which is incorporated herein by reference in its entirety.)

The disclosed methods relate to inhibition of the mitochondrial calcium uniporter (MCU) in cardiac pacemaker cells, which leads to heart rate stabilization. As disclosed herein, MCU inhibition by pacemaker-targeted delivery of genes encoding a dominant-negative (DN) MCU prevents heart rate increases by isoproterenol, which is a catecholamine agonist, without affecting basal heart rate. Thus, DN-MCU therapy is advantageous compared to other heart rate slowing drugs as it selectively prevents high heart rate without slowing basal heart rates.

Dominant negative mutants of MCU (e.g., DN-MCU or fragments thereof that bind to MCU and inhibit the activity of MCU) can be delivered by a vector. The term “vector” refers to some means by which DNA or RNA can be introduced into a host. There are various types of vectors including virus, plasmid, bacteriophages, cosmids, and bacteria. As used herein, a “viral vector” refers to recombinant viral nucleic acid that has been engineered to express a heterologous polypeptide (e.g., DN-MCU or fragments thereof that bind to MCU and inhibit the activity of MCU). The recombinant viral nucleic acid typically includes cis-acting elements for expression of the heterologous polypeptide.

Suitable vectors may include viral vectors comprising and expressing recombinant DNA encoding DN-MCU or fragments thereof that bind to MCU and inhibit the activity of MCU. The recombinant DNA typically is capable of being packaged into a helper virus that is capable of infecting a host cell. For example, the recombinant DNA acid may include cis-acting elements for packaging. Preferably, the helper virus is not replication competent, is attenuated, or at least does not cause disease. The helper virus may be genetically altered by modern molecular biological methods (e.g., restriction endonuclease and ligase treatment, and rendered less virulent than wild type), typically by deletion of specific genes. For example, the helper virus may lack a gene essential for production of infectious or virulent virus.

Suitable viral vectors may include, but are not limited to recombinant adenovirus, herpesvirus, retrovirus, or poxvirus vectors. Suitable adenoviral vectors may include “first generation” adenoviral vector. This group of adenoviral vectors is known in the art, and these viruses are characterized by being replication-defective. They typically have a deleted or inactivated E1 gene region, and preferably additionally have a deleted or inactivated E3 gene region. The first generation replication incompetent adenovirus vector used may be a serotype 5 adenovirus containing deletions in E1 (Ad5 base pairs 342-3523) and/or E3 (Ad5 base pairs 28133 to 30818). Those of skill in the art can easily determine the equivalent sequences for other serotypes, such as serotypes 2, 4, 12, 6, 17, 24, 33, 42, 31, 16. Adenoviral serotype 5 is preferred. However, it is envisioned that any adenovirus serotype can be used in the disclosed pharmaceutical composition, including non-human ones, as deletion of E1 genes should render all adenoviruses non-tumorigenic. The adenoviral vectors can be constructed using known techniques, such as those reviewed in Hitt et al, 1997 “Human Adenovirus Vectors for Gene Transfer into Mammalian Cells” Advances in Pharmacology 40:137-206, which is hereby incorporated by reference. In constructing the adenoviral vectors of this invention, it is often convenient to insert them in to a plasmid or shuttle vector. These techniques are known and described in Hitt et al. supra.

Patients at risk for pacemaker cell dysfunction could be treated with DN-MCU or fragments thereof that bind to MCU and inhibit the activity of MCU by gene “painting” at the time of thoracic surgery or by minimally invasive mediastinoscopy. Gene painting has been known since 2005. (See Kikuchi et al., Targeted Modification of Atrial Electrophysiology by Homogeneous Transmural Atrial Gene Transfer, Circulation 2005; 111:264-270, the content of which is incorporated herein by reference in its entirety). For example, patients may be treated by gene “painting” of recombinant nucleic acid or a viral vector expressing DN-MCU or fragments thereof that bind to MCU and inhibit the activity of MCU.

### EXAMPLES

The following examples are illustrative and are not intended to limit the disclosed subject matter.

### Example 1

Reference is made to Wu et al., “The mitochondrial uniporter controls fight or flight heart rate increases, Nature Commun. 2015 January 20; 6:6081, the content of which is incorporated herein by reference in its entirety.

The Mitochondrial Uniporter Controls Fight or Flight Heart Rate Increases

Catecholamine agonists trigger physiological fight or flight increases in heart rate but the metabolic pathway(s) supplying ATP for increasing heart rate are unknown.^{1,2} Here we show that Ca²⁺ entry through the recently discovered mitochondrial Ca²⁺ uniporter (MCU)^{3,4} is essential for telegraphing enhanced metabolic demand to pacemaker cell mitochondria and promoting oxidative phosphorylation. We found that isoproterenol (ISO) stimulates oxidative phosphorylation by the MCU pathway in cardiac pacemaker cells to fuel the activity of the sarcoplasmic-endoplasmic reticulum Ca²⁺ ATPase (SERCA2a), which is required for reloading intracellular Ca²⁺ stores and sustaining fight or flight heart rate increases. We developed mice with myocardial-targeted transgenic expression of a dominant negative (DN) MCU and used pacemaker-targeted DN-MCU gene therapy in wild type mice to show loss of MCU function selectively impairs ISO stimulated heart rate responses in vivo and ex vivo, without affecting basal heart rates. Inhibition of mitochondrial Ca²⁺ entry prevented increased oxidative phosphorylation, enhanced SERCA2a activity and physiological rate responses in cardiac pacemaker cells exposed to ISO. Dialysis of cardiac pacemaker cells with exogenous ATP rescued the fight or flight response to ISO despite MCU inhibition but ATP dialysis was ineffective after SERCA2a inhibition, identifying SERCA2a as a critical control point downstream of MCU for heart rate increases and a preferential sink for mitochondrially-sourced ATP. Our findings highlight a previously unrecognized subcellular mechanism for catecholamine-triggered heart rate increases and provide insight into a role for mitochondrial Ca²⁺ as a metabolic second messenger required for the physiological tight or flight stress response.⁵ These results define the MCU as an essential activator of a metabolic pathway for heart rate control and suggest that MCU inhibition in cardiac pacemaker cells has therapeutic potential to selectively prevent excessive heart rates.

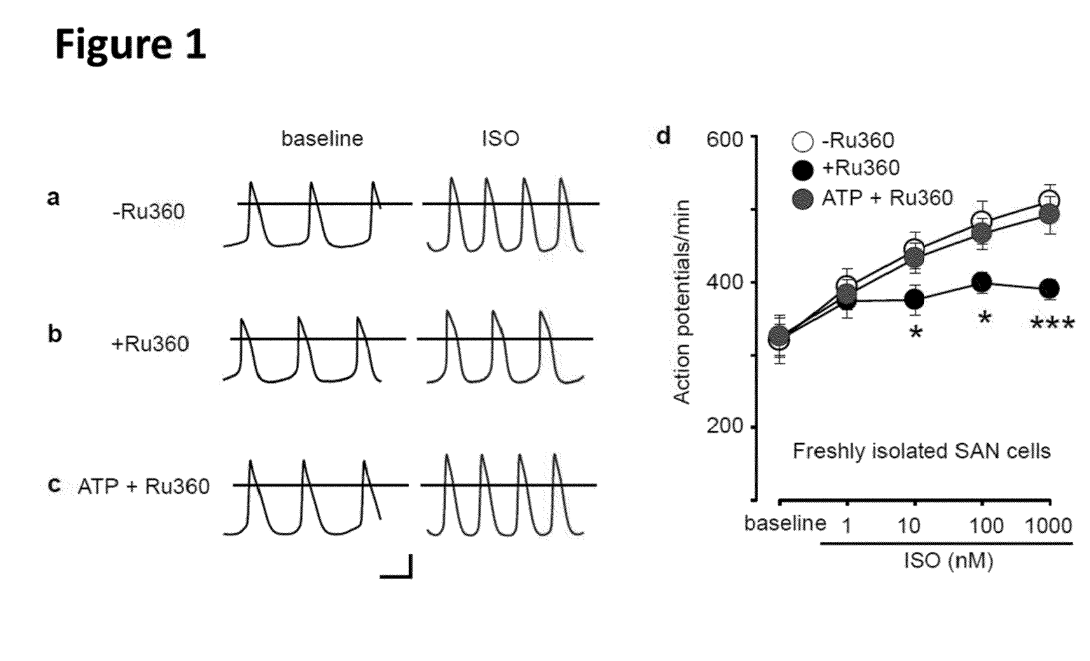

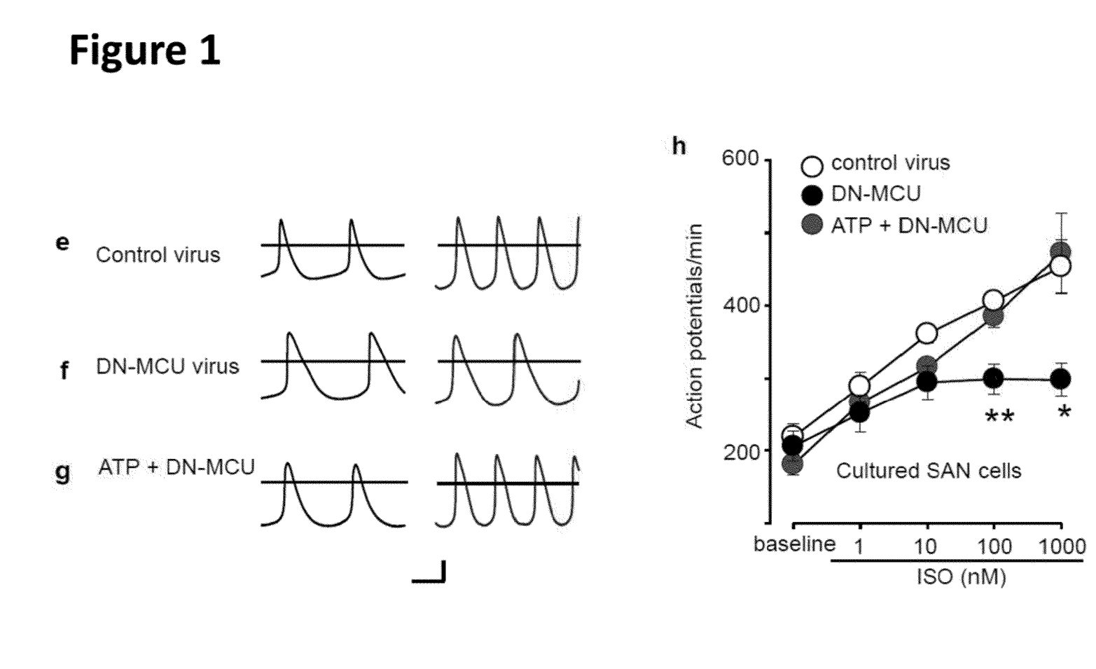

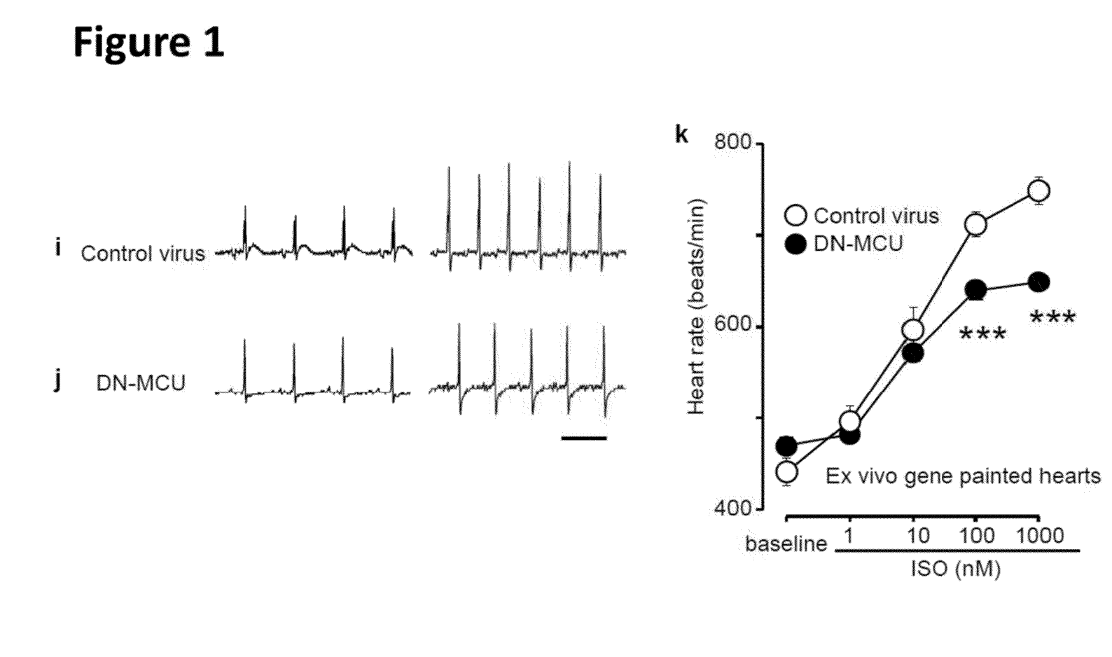

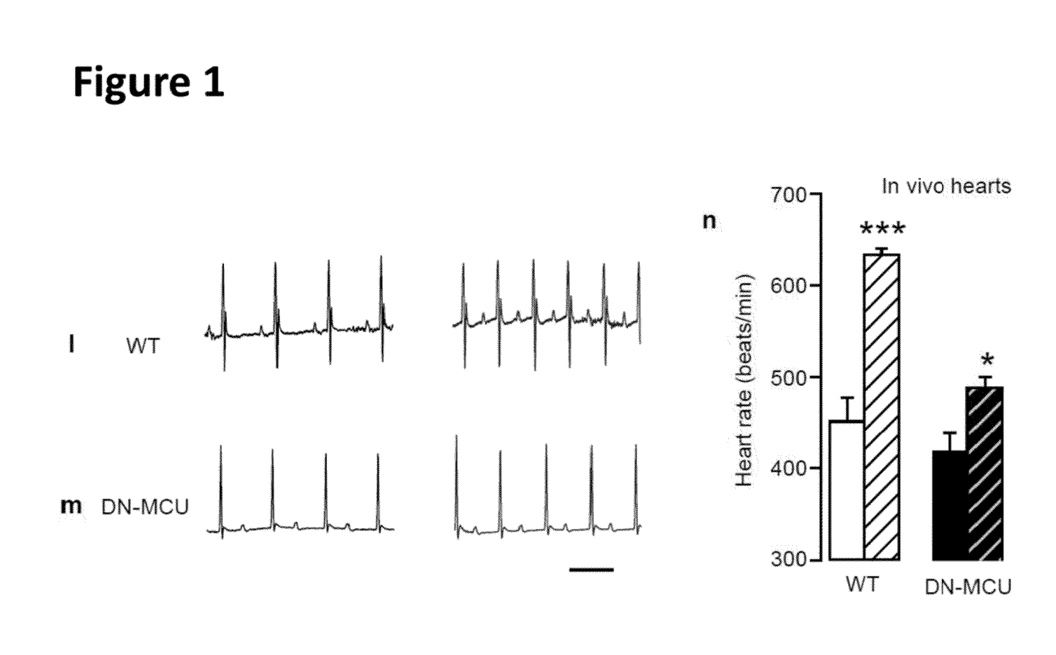

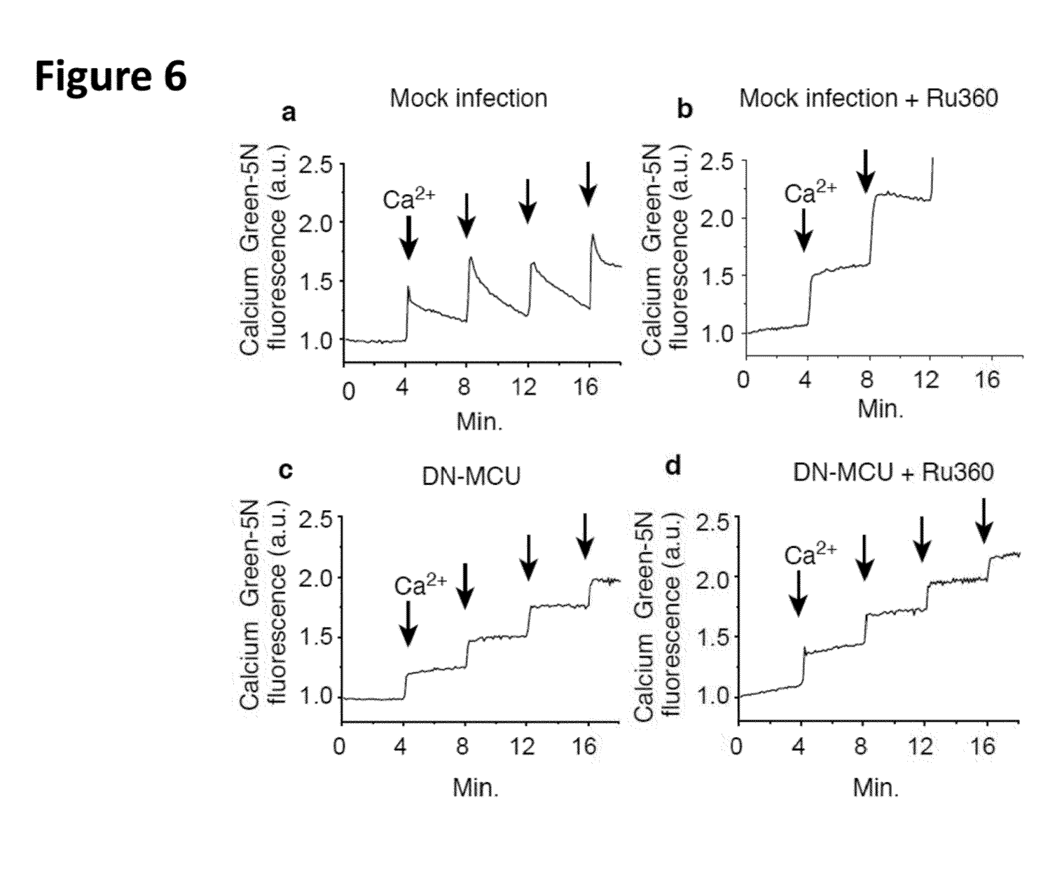

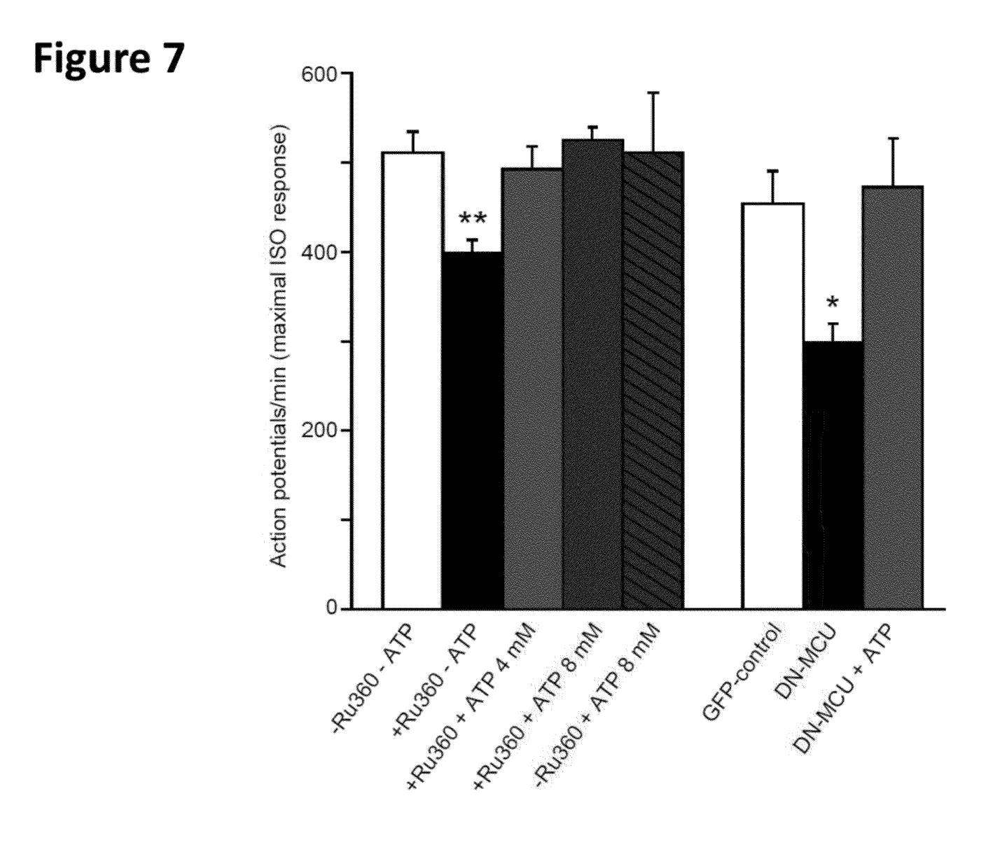

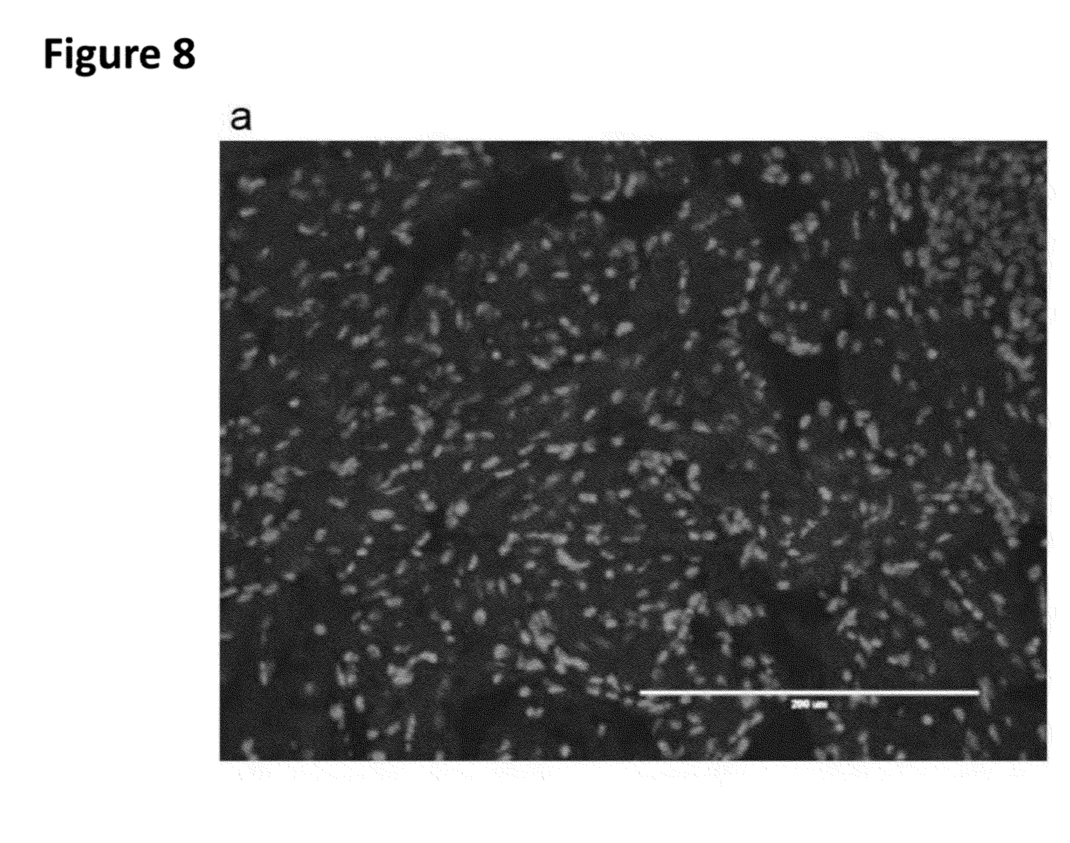

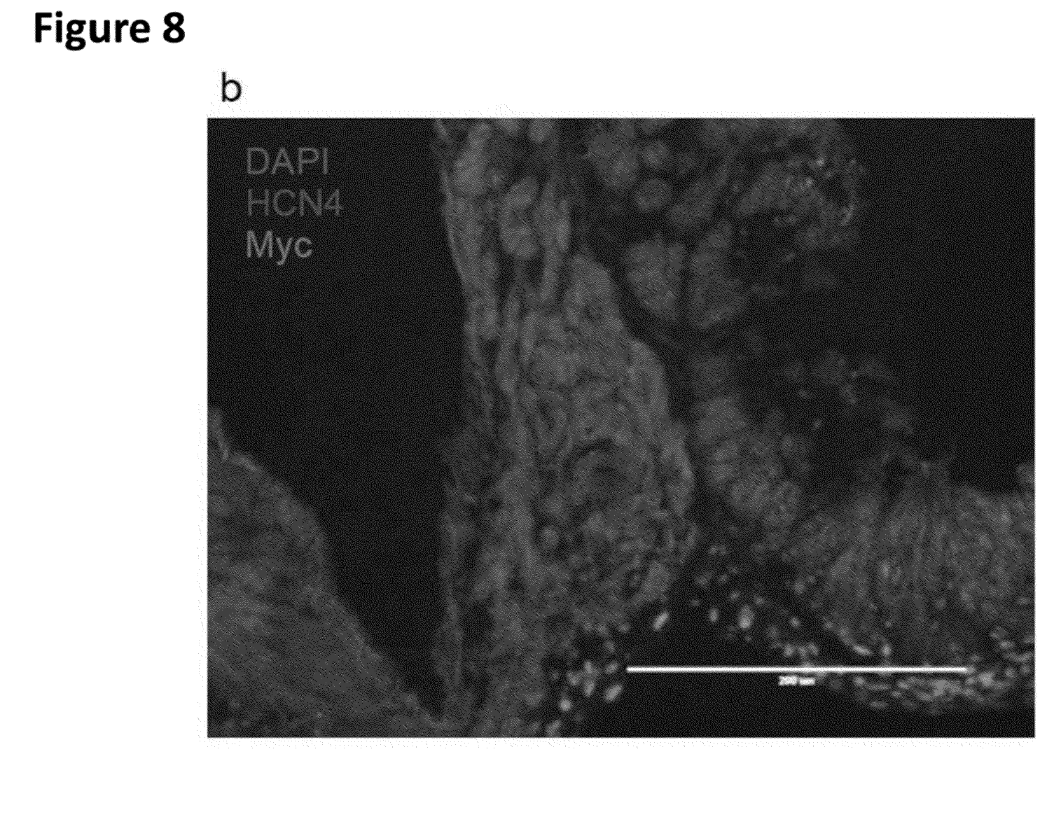

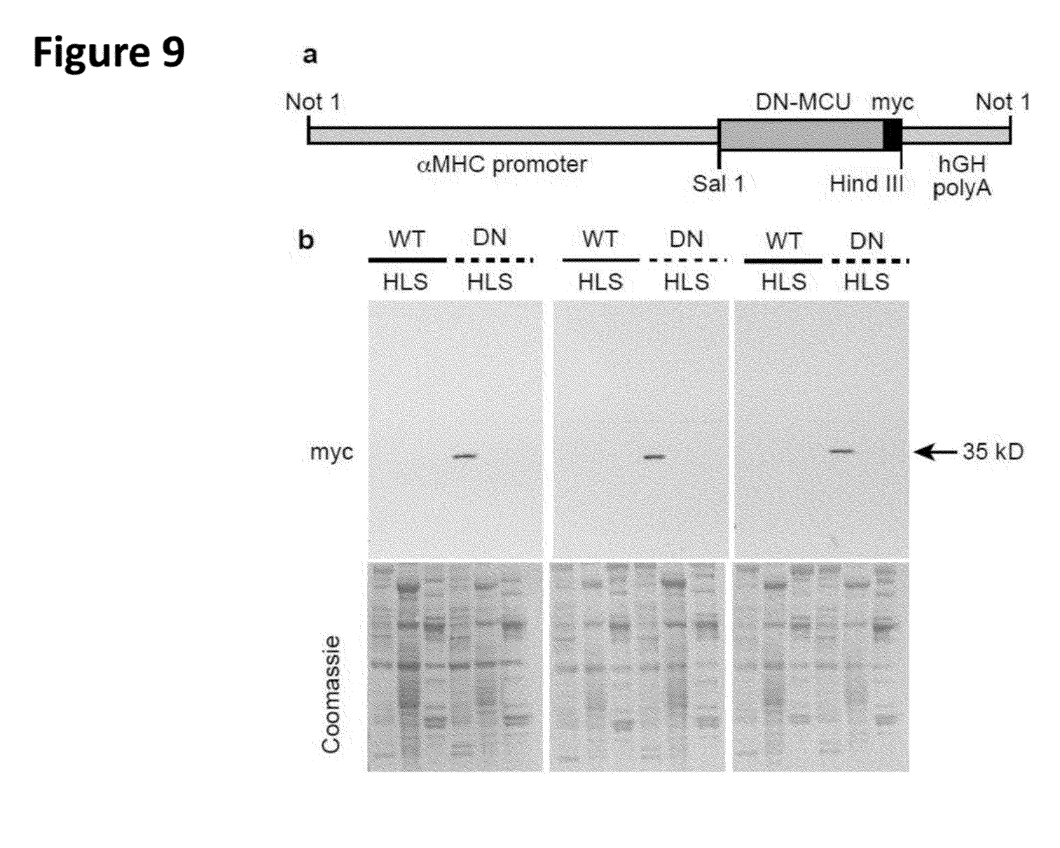

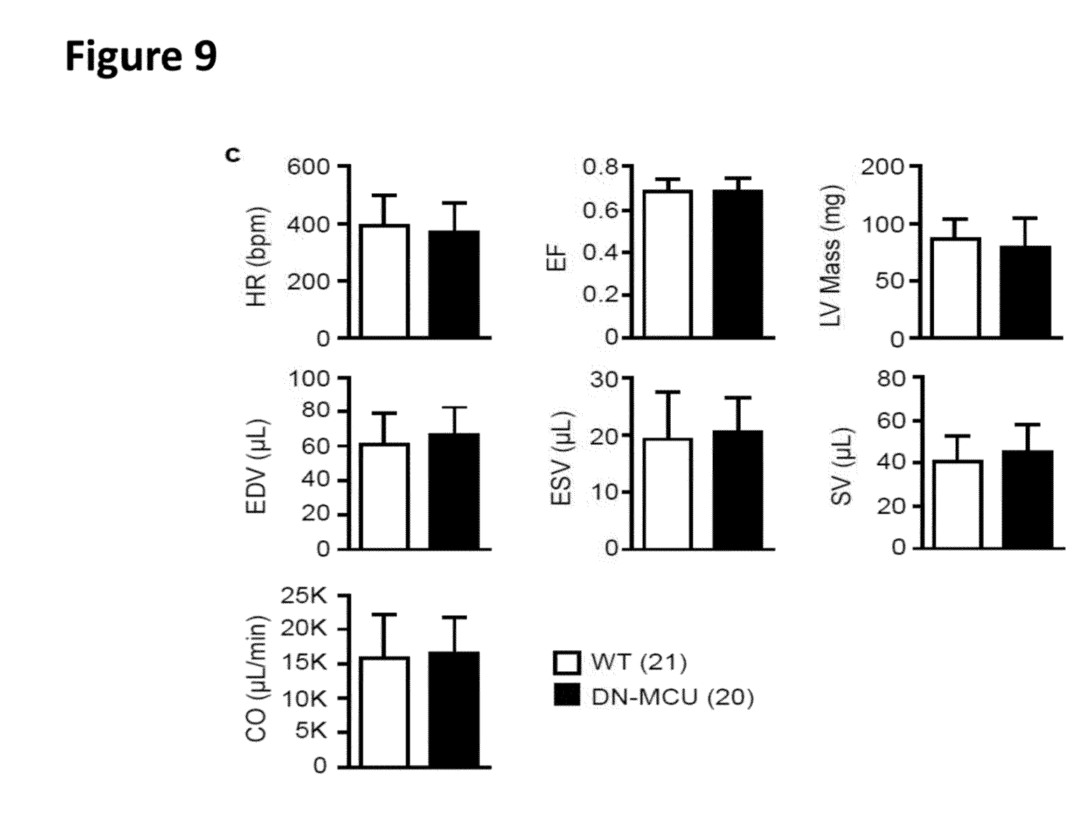

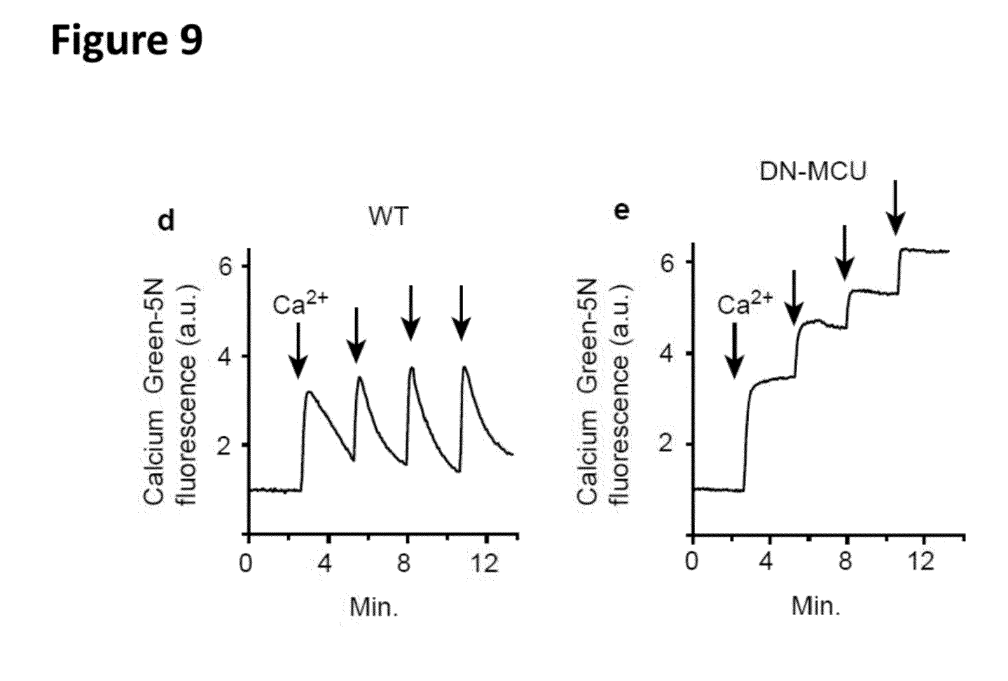

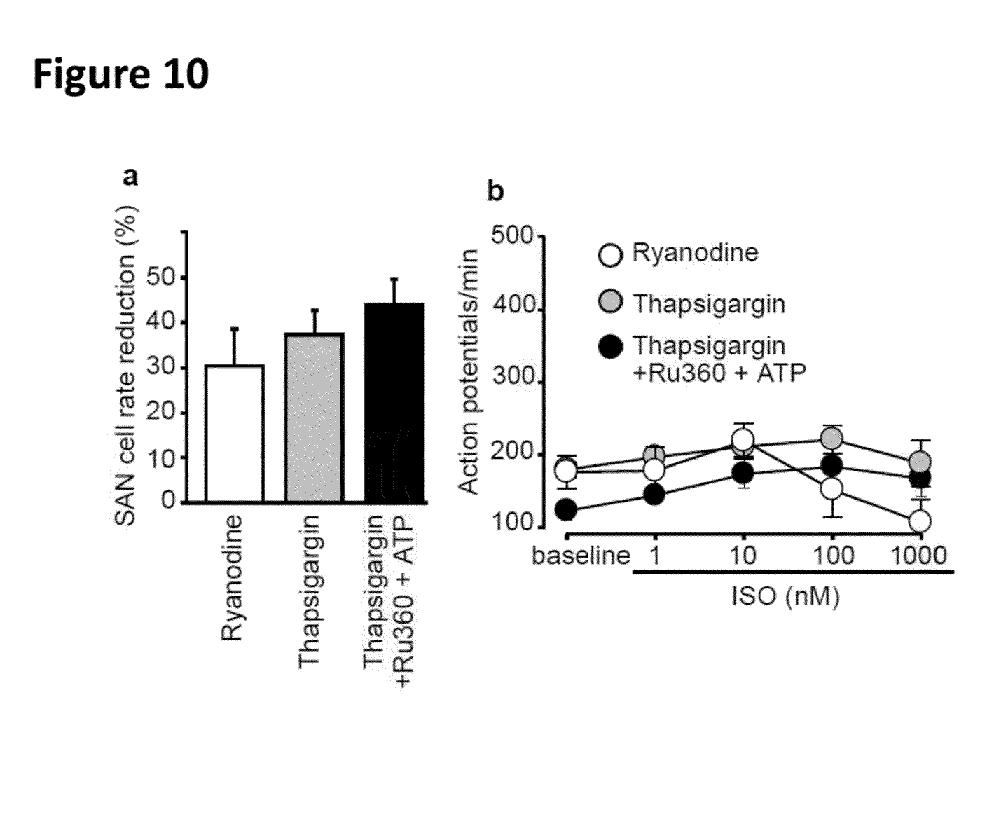

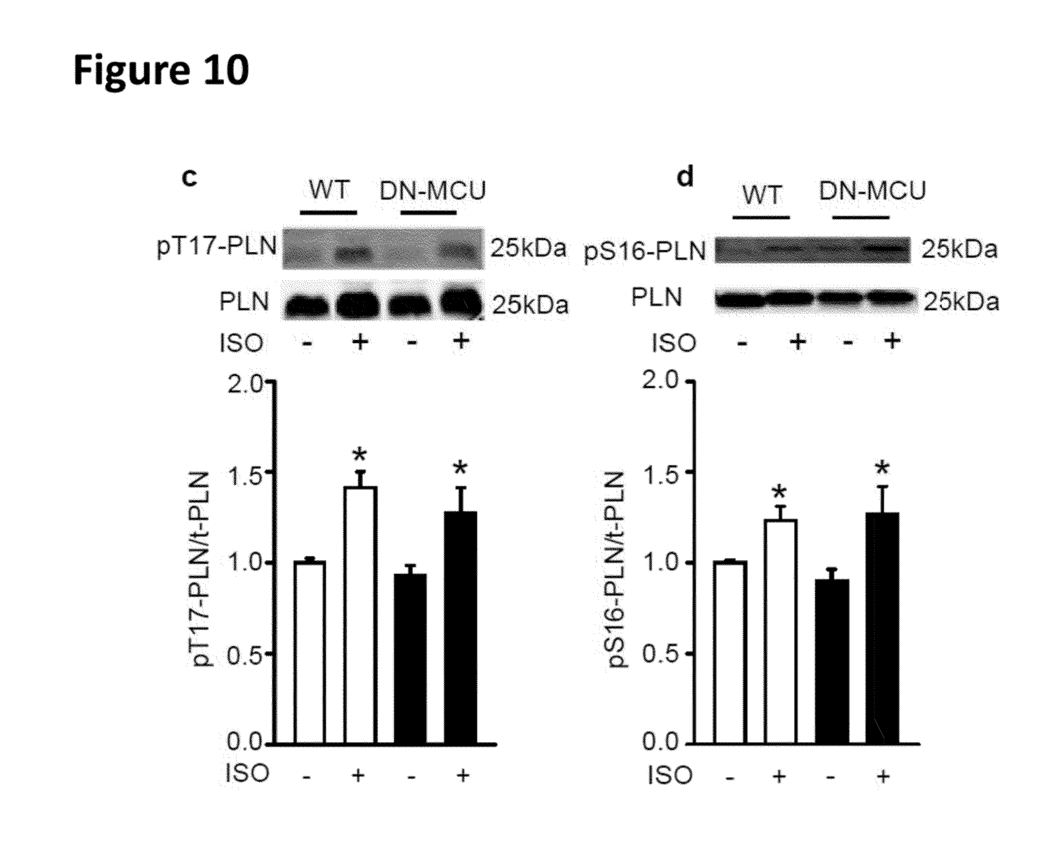

Isolated cardiac sinoatrial nodal (SAN) pacemaker cells spontaneously generate action potentials under basal conditions, in the absence of catecholamine stimulation. The rate of action potential initiation is increased with ISO, a catecholamine β adrenergic receptor agonist, in a concentration-dependent manner (FIG. 1a-d).⁶ We found that SAN cells dialyzed with Ru360 (5 μM), an MCU antagonist, had significantly reduced action potential frequency increases to ISO (FIG. 1b and d) compared to SAN cells without Ru360 (FIG. 1a and d). The inhibitory effect of Ru360 on SAN cell action potential frequency responses to ISO was reversed by co-dialysis with ATP (4 mM), a concentration present in heart cells (FIG. 1c and d).⁷ In contrast, neither Ru360 nor ATP had any affect on basal SAN cell rates and ATP dialysis had no affect on SAN rate responses to ISO (FIG. Sa and b), indicating that basal heart rate was independent of an MCU pathway and that exogenous ATP did not affect Ru360-independent cellular processes important for accelerating heart rate. We next corroborated our results with Ru360 dialysis by infecting SAN cells with adenovirus encoding a DN form of MCU (DN-MCU), containing pore domain charge reversal mutations that prevent MCU mediated mitochondrial Ca²⁺ entry^{3,4}. Both Ru360 and DN-MCU were similarly effective at preventing mitochondrial Ca²⁺ entry (FIG. 6) and, like intracellular dialysis with Ru360, DN-MCU expression in SAN cells prevented ISO induced rate increases (FIG. 1f and h). The DN-MCU dependent loss of the ISO rate response was rescued by co-dialysis with ATP (FIG. 1g and h), mirroring findings with MCU inhibition by Ru360 (FIG. 1d). Cultured SAN cells, with and without DN-MCU expression, had slower basal spontaneous rates than freshly isolated counterparts but exhibited preserved rate increases in response to ISO (FIG. 7). The rescue of physiological responses to ISO after MCU inhibition by ATP dialysis suggested that MCU activity was an upstream event required to supply adequate ATP for increasing heart rate. The selective control of pacemaker cell rate increases by MCU inhibition suggested that MCU inhibition could provide a novel approach to controlling heart rate increases without slowing resting heart rates. In order to test this concept in hearts, we used a gene painting approach to deliver adenovirus expressing DN-MCU or eGFP to the SAN in vivo.⁸ One week after SAN gene painting we verified SAN targeted gene expression (FIG. 8) and measured heart rate responses to ISO (FIG. 1i-k). We found DN-MCU expression significantly and selectively reduced heart rate increases to ISO without affecting spontaneous heart rates in the absence of ISO (FIGS. 1j and k). We next developed transgenic mice with myocardial DN-MCU expression (FIG. 9a). These DN-MCU mice had hearts with normal chamber size and function (FIG. 9b), complete loss of mitochondrial Ca²⁺ uptake in myocardial cells (FIGS. 9c and d) and exhibited resting heart rates similar to wild type littermate controls. However, DN-MCU mice showed a dramatic loss of the heart rate response to ISO compared to wild type littermate controls (FIG. 10). Taken together, these data showed SAN rate responses to ISO required MCU, while basal SAN automaticity was independent of MCU, and that MCU inhibition could prevent or reduce excessive increases in heart rate.

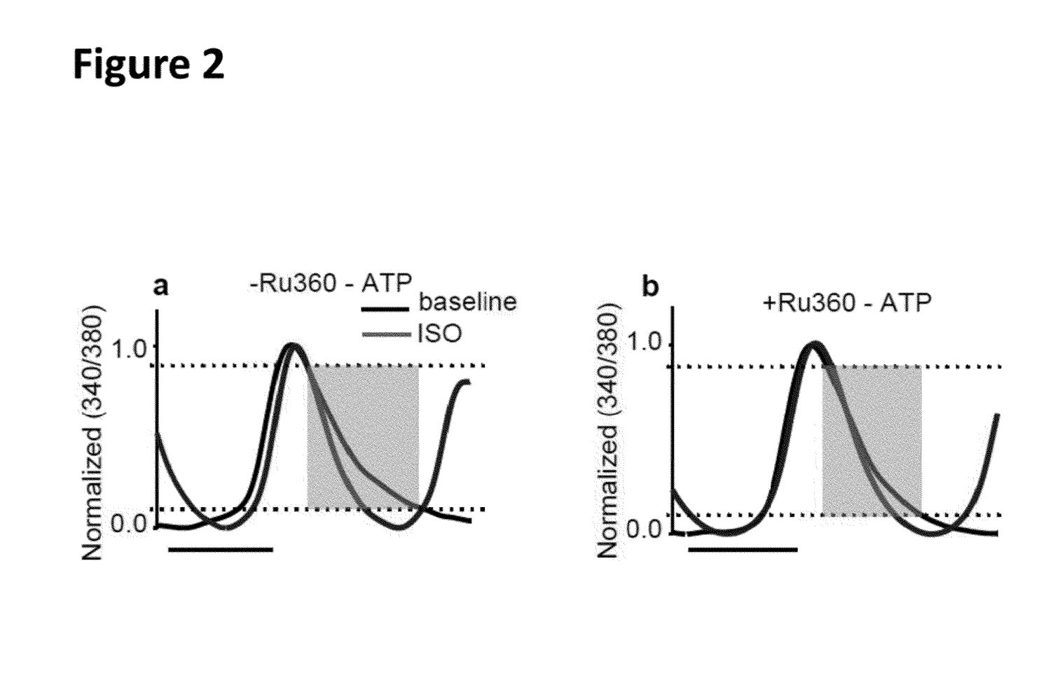

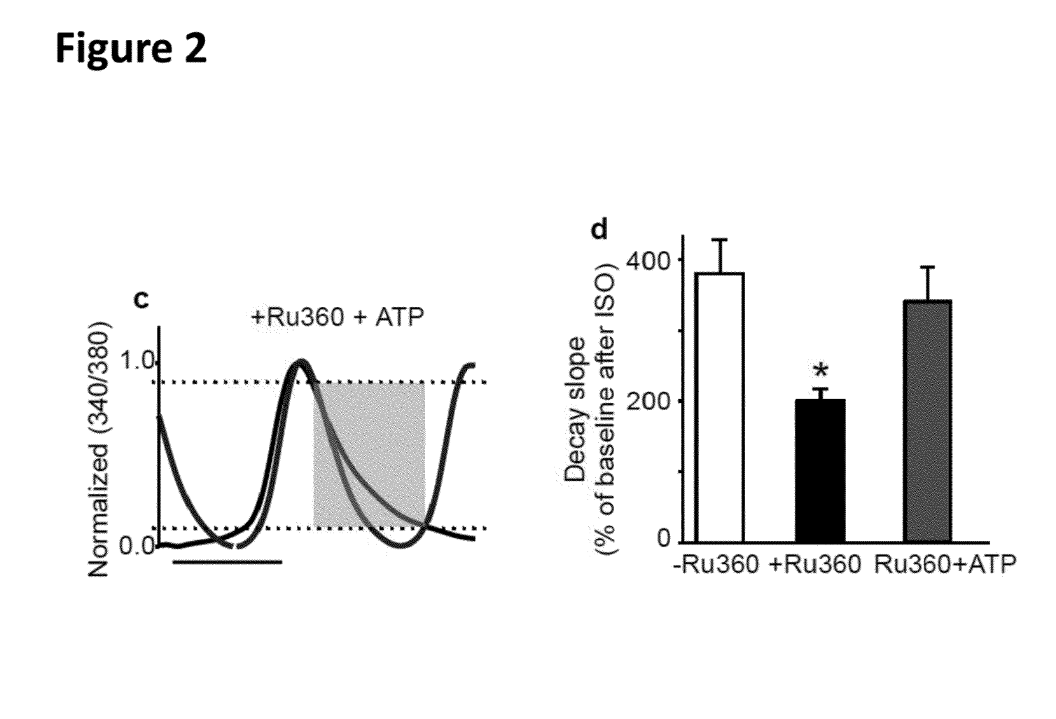

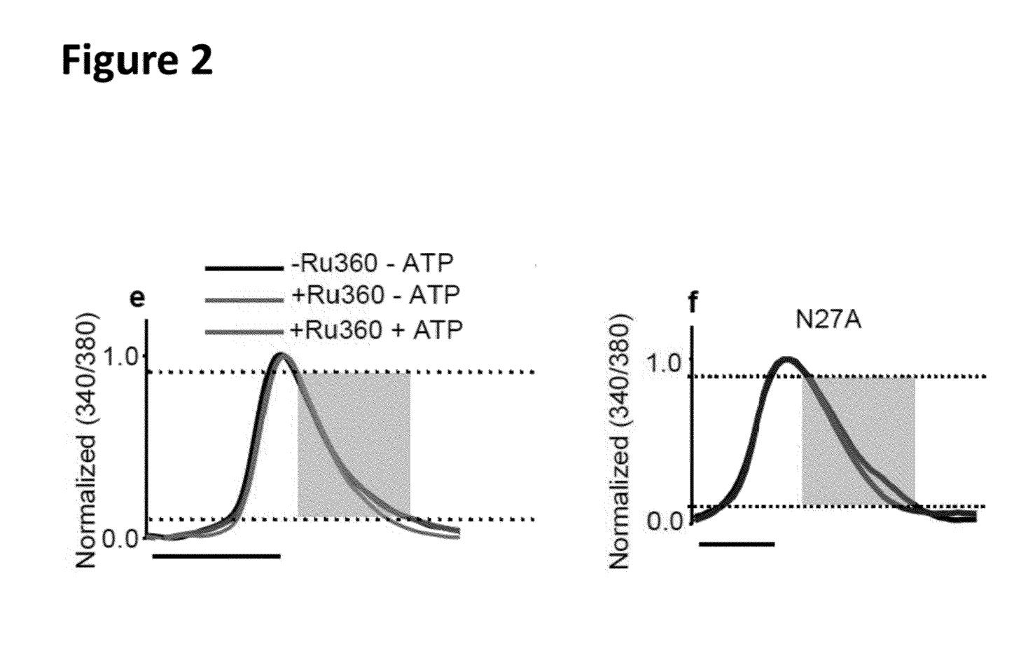

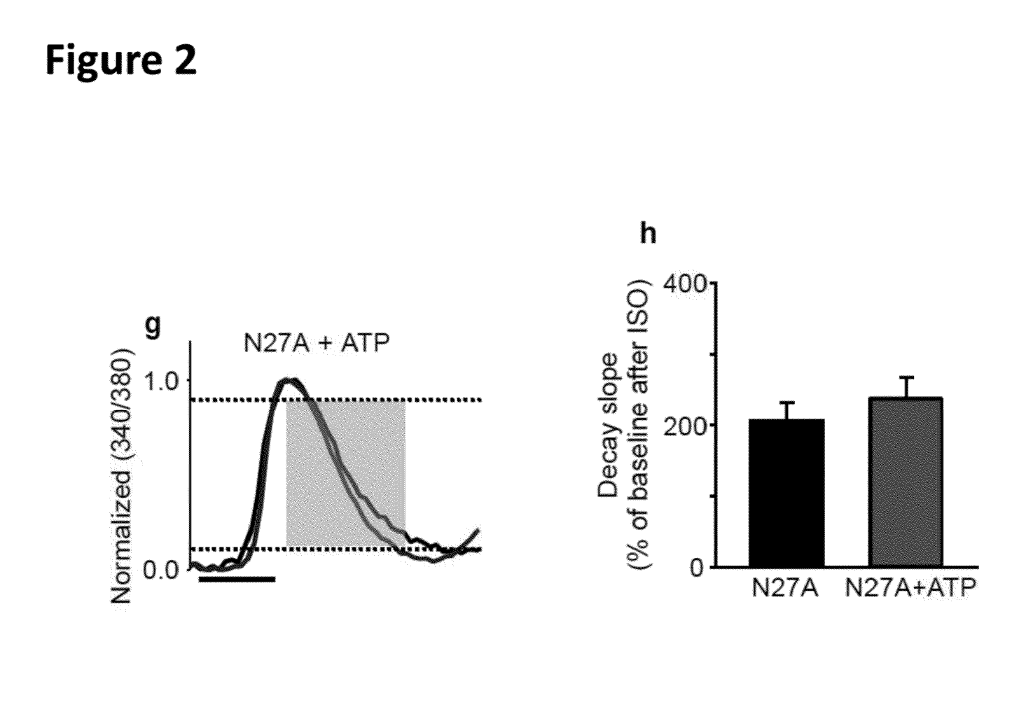

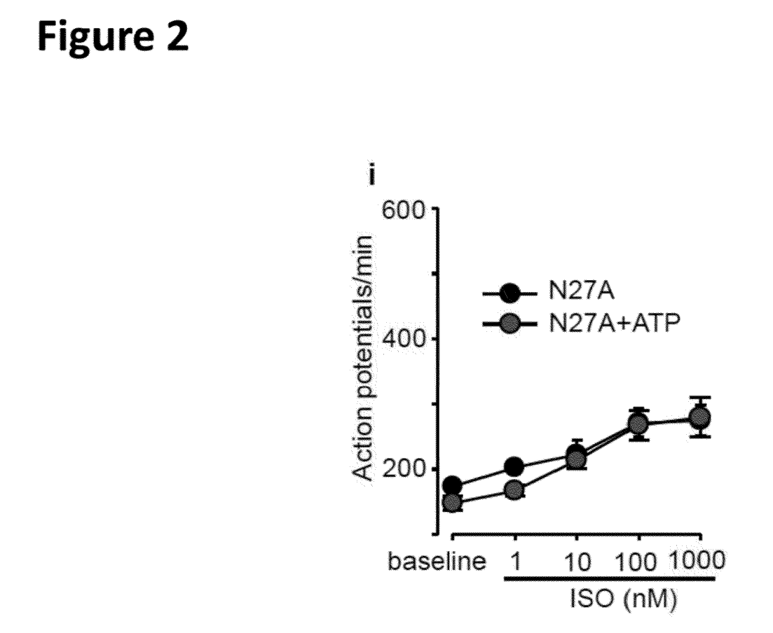

Catecholamine stimulation increases heart rate by enhancing SAN cell membrane inward current and shortening the time between action potential firing.⁹ Release of intracellular Ca²⁺ from the sarcoplasmic reticulum (SR) provides the electrochemical driving force for the cell membrane Na⁺/Ca²⁺ exchanger inward current (I_{NCX}) in SAN cells.¹⁰ Elimination of SR Ca²⁺ release by the toxin ryanodine or SERCA2a inhibition by thapsigargin (FIGS. 10a and b) significantly reduced the SAN activity response to ISO, findings that demonstrate the required connection between SR Ca²⁺ release and physiological SAN cell acceleration.¹¹ SERCA2a activity requires ATP to pump cytoplasmic Ca²⁺ to the SR lumen¹², so we measured the rate of decay of the cytoplasmic Ca²⁺ concentration ([Ca²⁺]_{cyto}) using a fluorescent indicator (Fura 2 AM, 0.1 μM) to test if SERCA2a was a sink for ATP produced by an Ru360-sensitive process. ISO significantly increased the rate of decline in [Ca²⁺]_{cyto} compared to SAN cells in control bath solution, reflecting enhanced activity of SERCA2a (FIG. 2a).¹³ Ru360 dialysis slowed the decline in [Ca²⁺]_{cyto} after ISO (FIG. 2b), while co-dialysis of ATP (4 mM) with Ru360 restored the rate of [Ca²⁺]_{cyto} decline to ISO-stimulated values present in the absence of Ru360 (FIGS. 2c and d). However, Ru360 did not slow the decline in [Ca²⁺]_{cyto} in the absence of ISO stimulation (FIG. 2e), consistent with the lack of effect of Ru360 on SERCA2a activity or basal SAN cell action potential frequency. In contrast to the effect of ATP dialysis on SAN cells exposed to Ru360, ATP dialysis did not significantly increase the rate of [Ca²⁺]_{cyto} decline (FIG. 2f-h) or SAN action potential frequency (FIG. 2i) in SAN cells isolated from mice expressing a super inhibitory mutant form of phospholamban (N27A)¹⁴ that constrains SERCA2a despite ISO stimulation. The results show that ISO increases SAN cell rates by actions that require MCU and SERCA2a. We considered the possibility MCU inhibition was somehow affecting the ability of ISO to enhance phospholamban phosphorylation, which reduces the inhibitory actions of phospholamban on SERCA2a.¹⁵ We found that atrial tissues from DN-MCU and wild type littermates had similar increases in phosphorylation of phospholamban (FIG. 10c and d), suggesting that MCU inhibition did not interfere with SERCA2a activity by actions on phospholamban nor did DN-MCU expression promiscuously affect downstream signaling actions of ISO. We interpreted the rescue of ISO responses by exogenous ATP after elimination of MCU-mediated Ca²⁺ entry but not after SERCA2a inhibition to suggest that MCU contributes to ATP synthesis targeted for SERCA2a consumption during physiological stress.

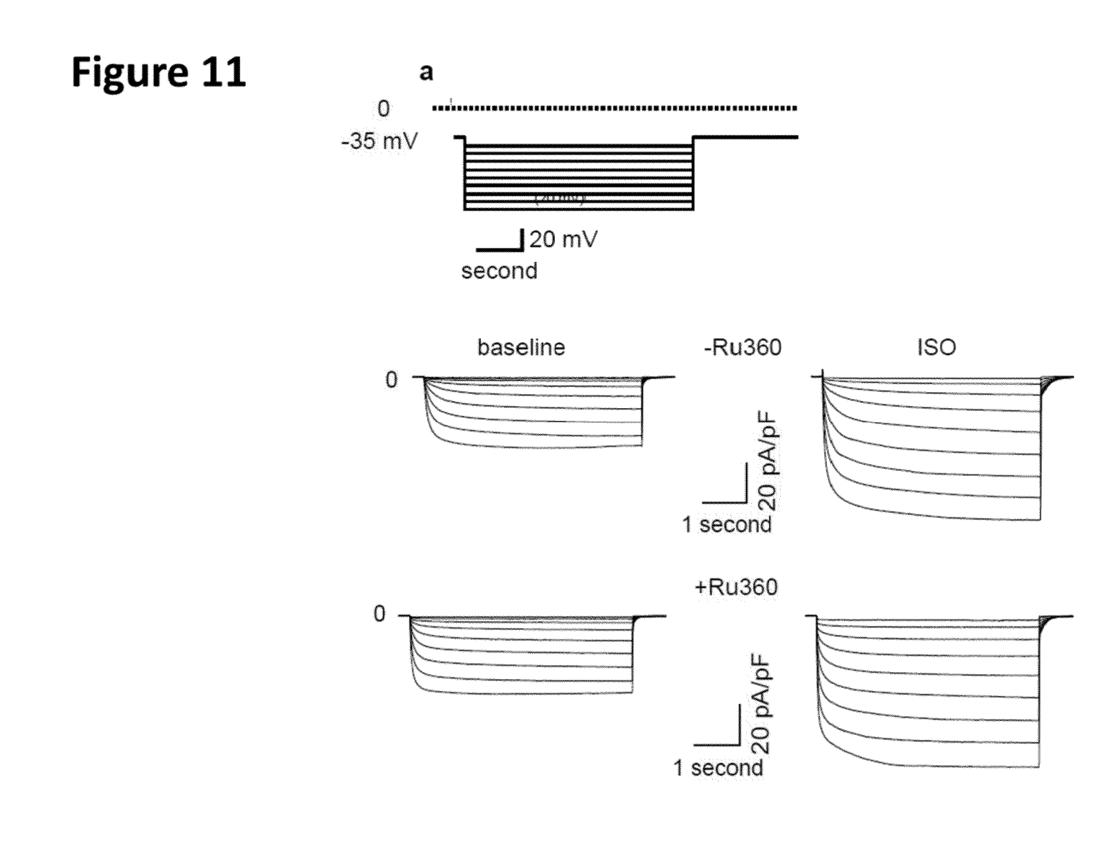

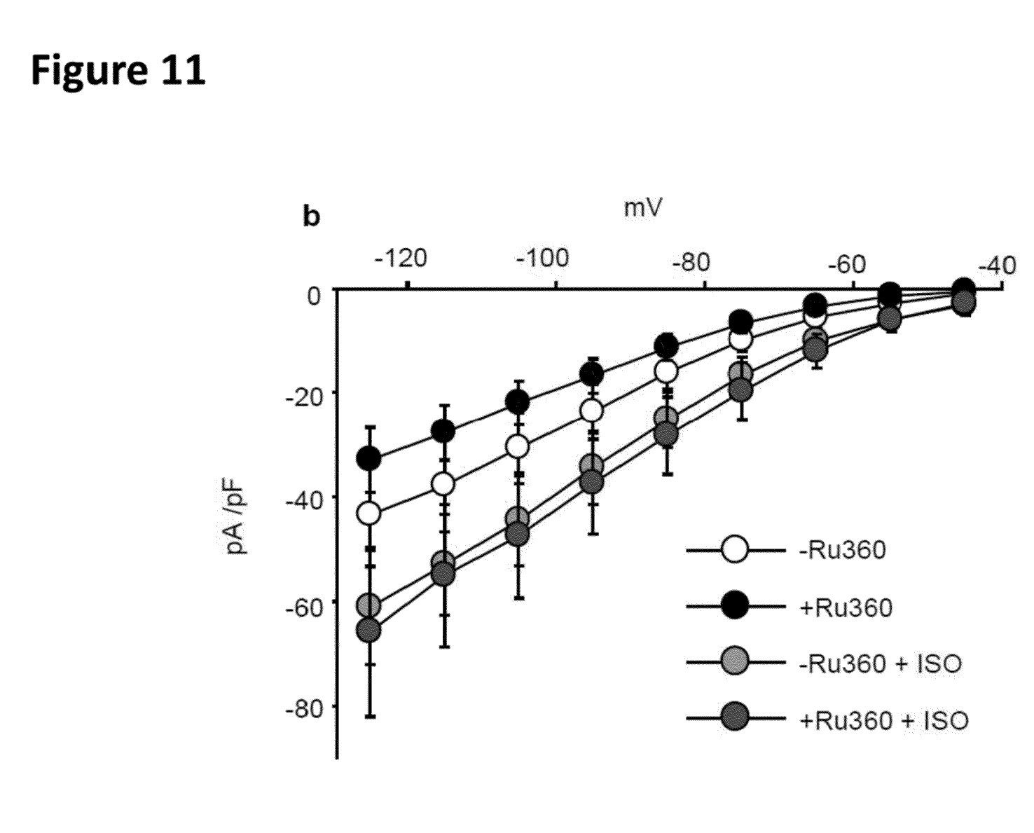

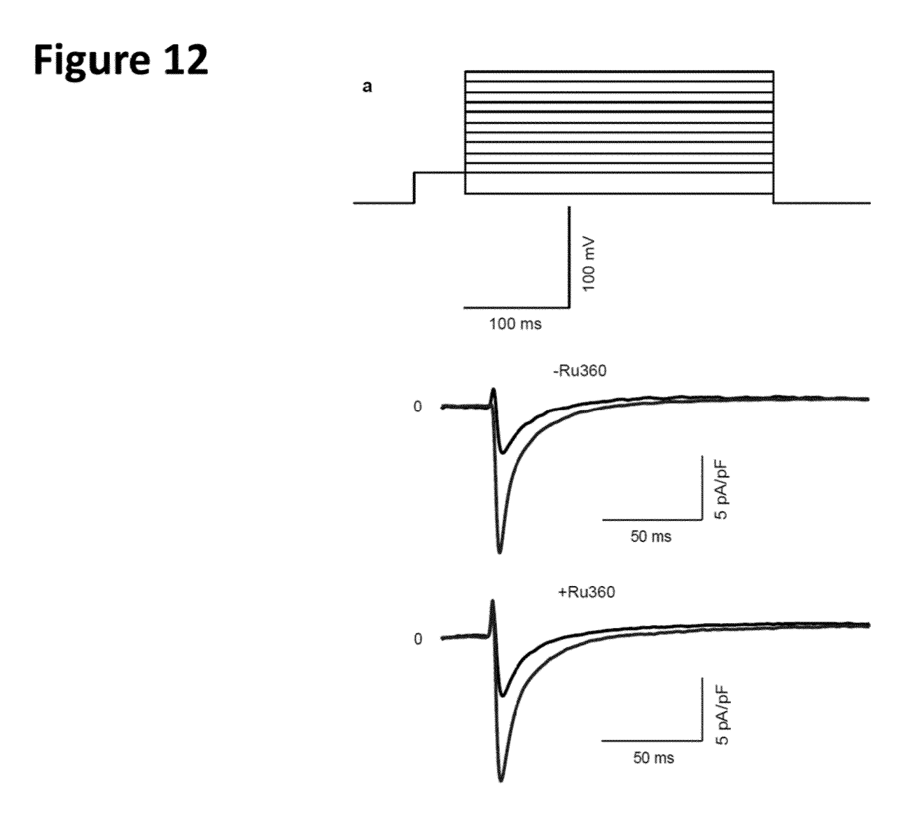

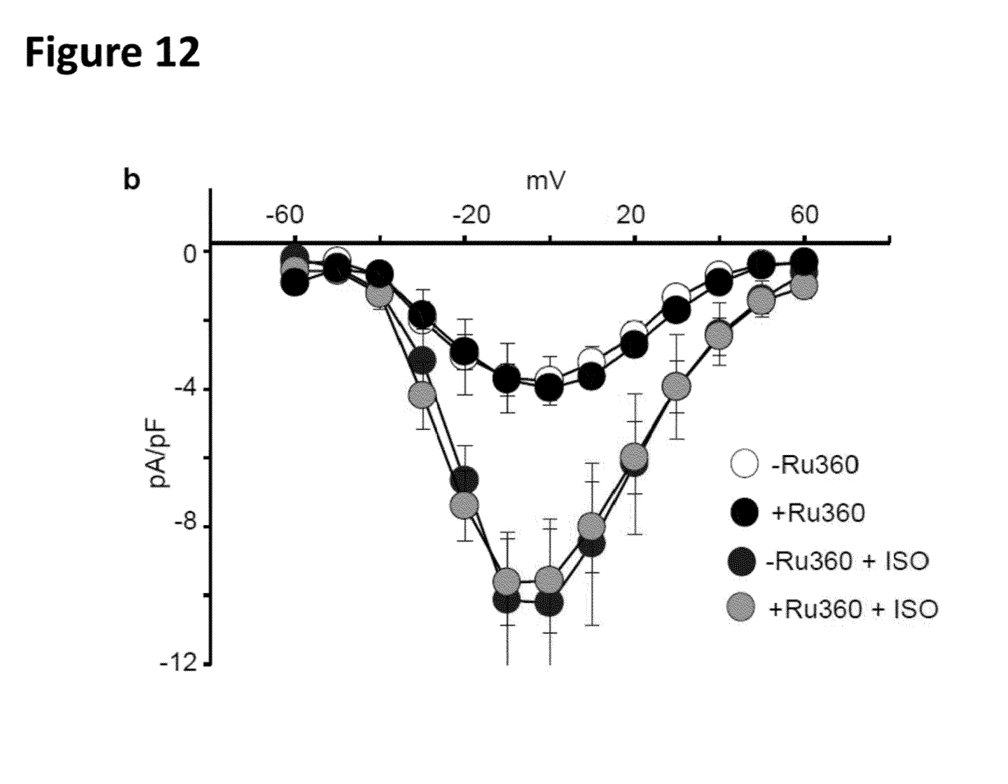

Because heart rate is responsive to multiple ionic currents⁹, we next asked if the MCU pathway affected SERCA2a-independent pacing mechanisms. ISO may contribute to heart rate increases by augmenting an ATP and 3′-5′-cyclic adenosine monophosphate (cAMP) dependent cell membrane ion channel (HCN4) inward current (I_{f}).¹⁶ We found that I_{f} responses to ISO were not reduced by Ru360 dialysis (FIG. 11). The lack of effect of Ru360 on I_{f} suggested that Ru360 did not reduce cAMP nor ATP availability globally in SAN cells below a threshold necessary to increase I_{f}. We also measured Ca_{V}l L-type Ca²⁺ current (I_{Ca}), an SAN cell membrane inward current enhanced by ISO through ATP-mediated phosphorylation.¹⁷ Similar to our findings with I_{f}, ISO-induced I_{Ca} increases were not impaired by Ru360 (FIG. 12). These findings were consistent with a model where selective loss of heart rate acceleration after ISO by Ru360 was primarily or exclusively related to actions on SERCA2a.

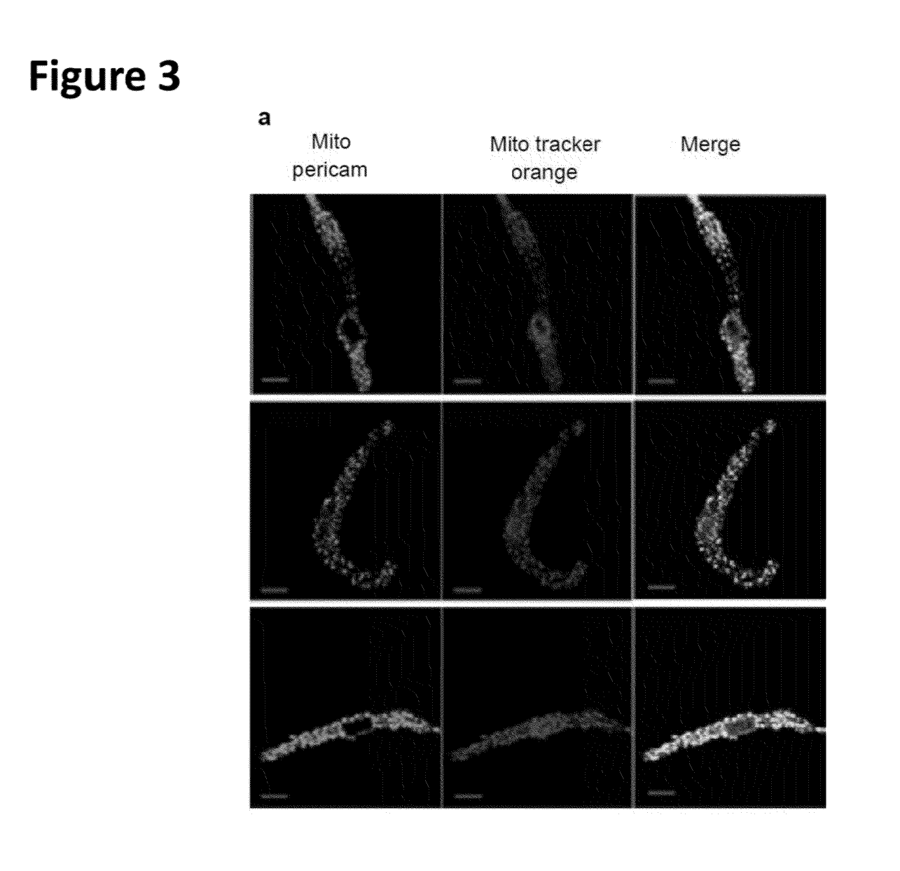

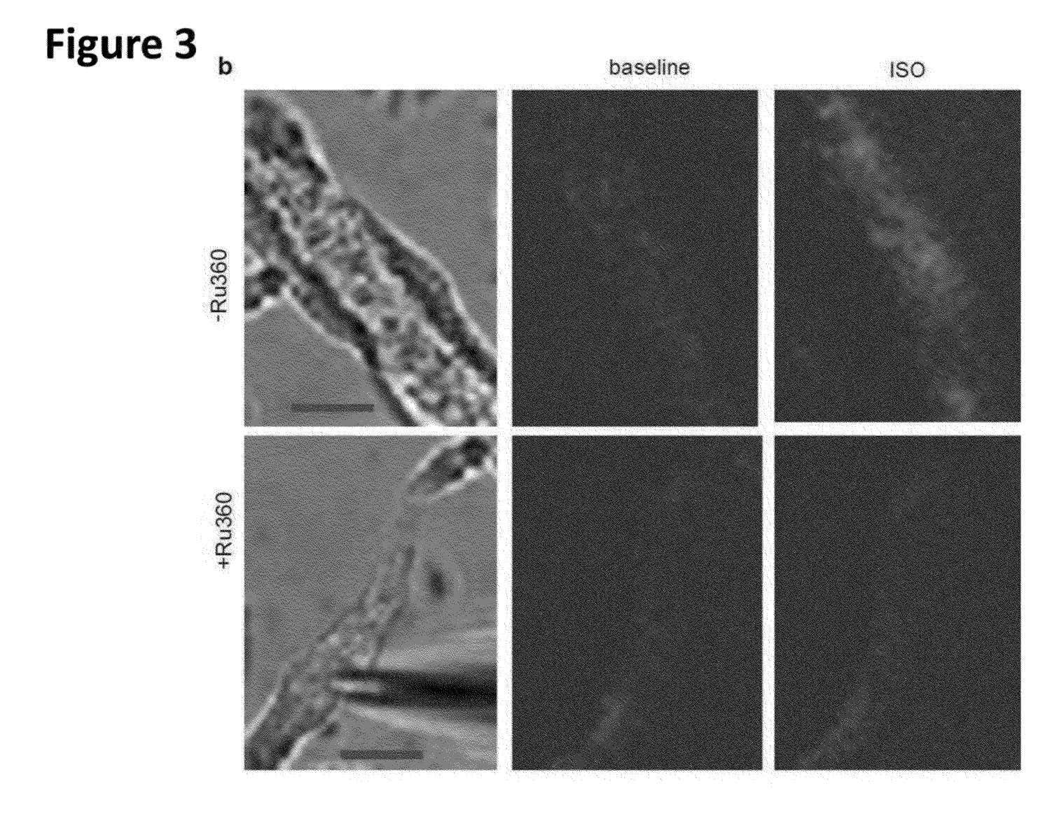

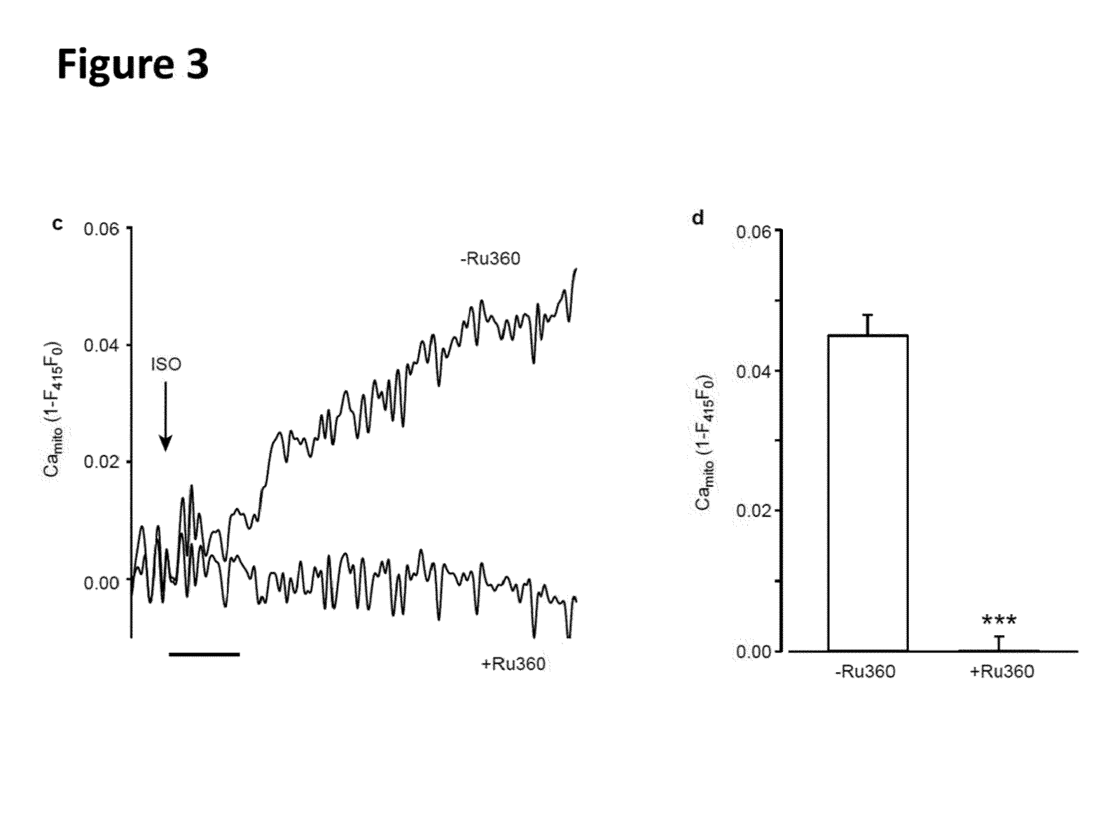

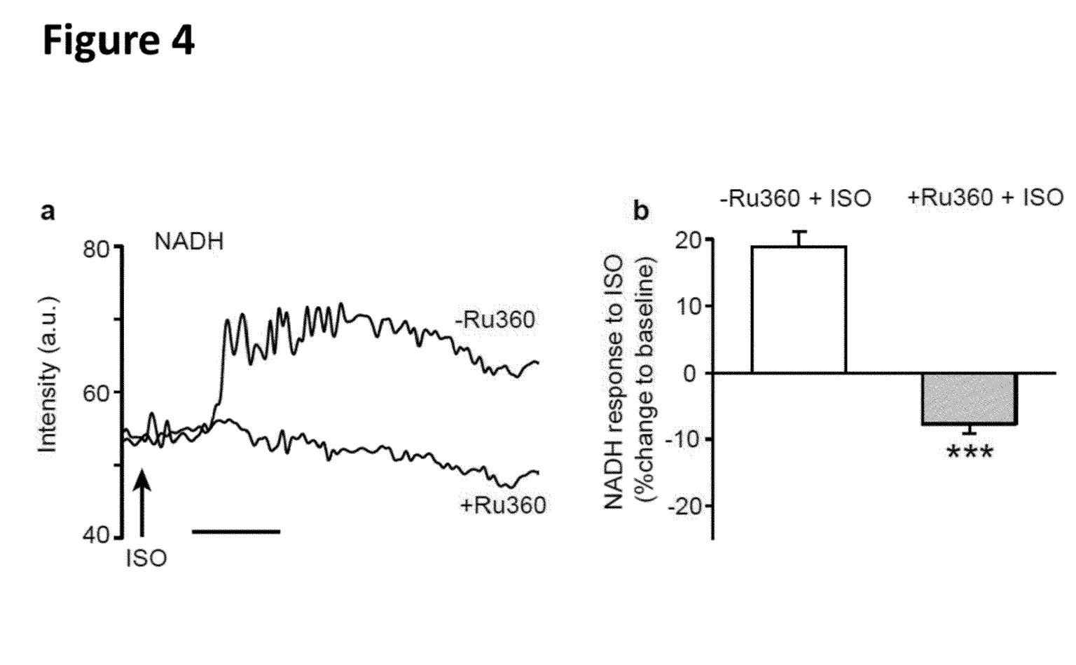

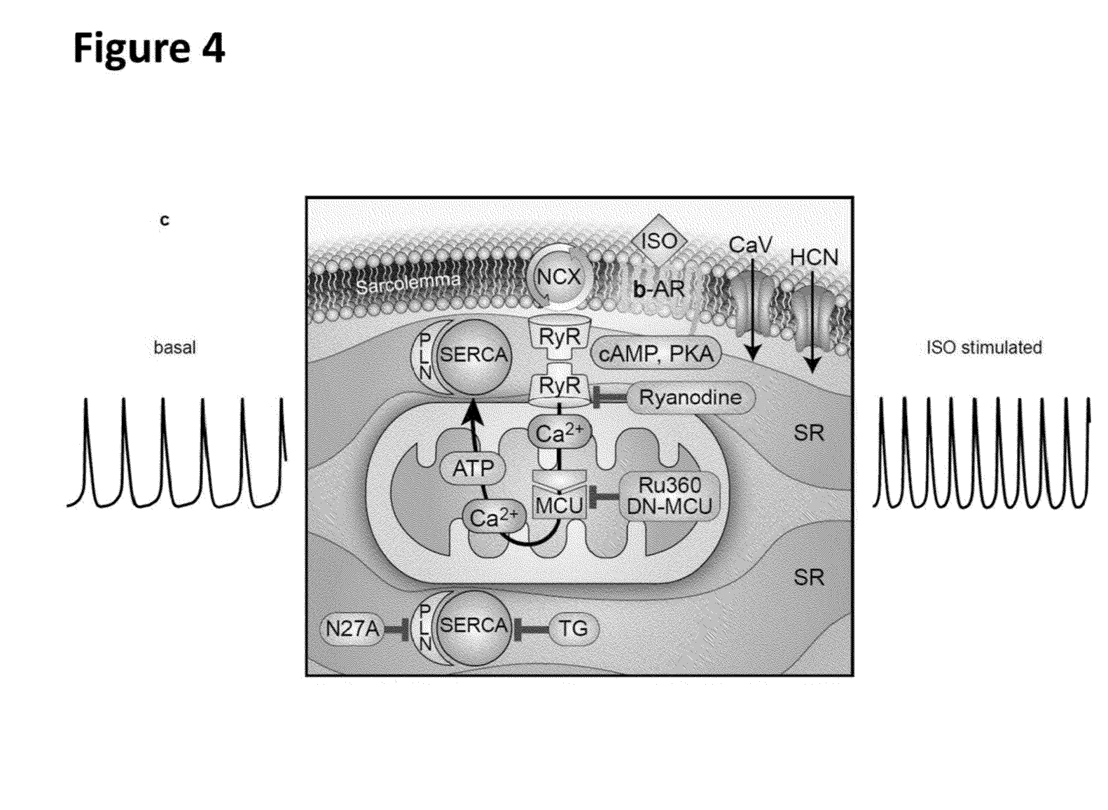

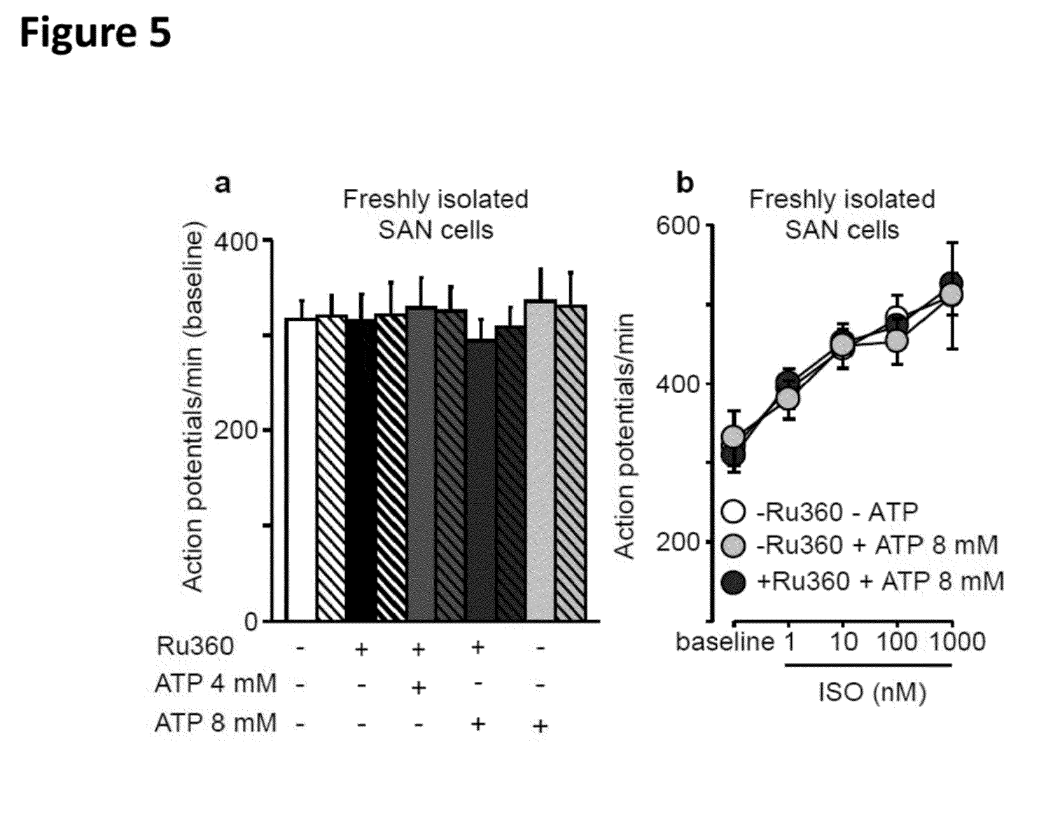

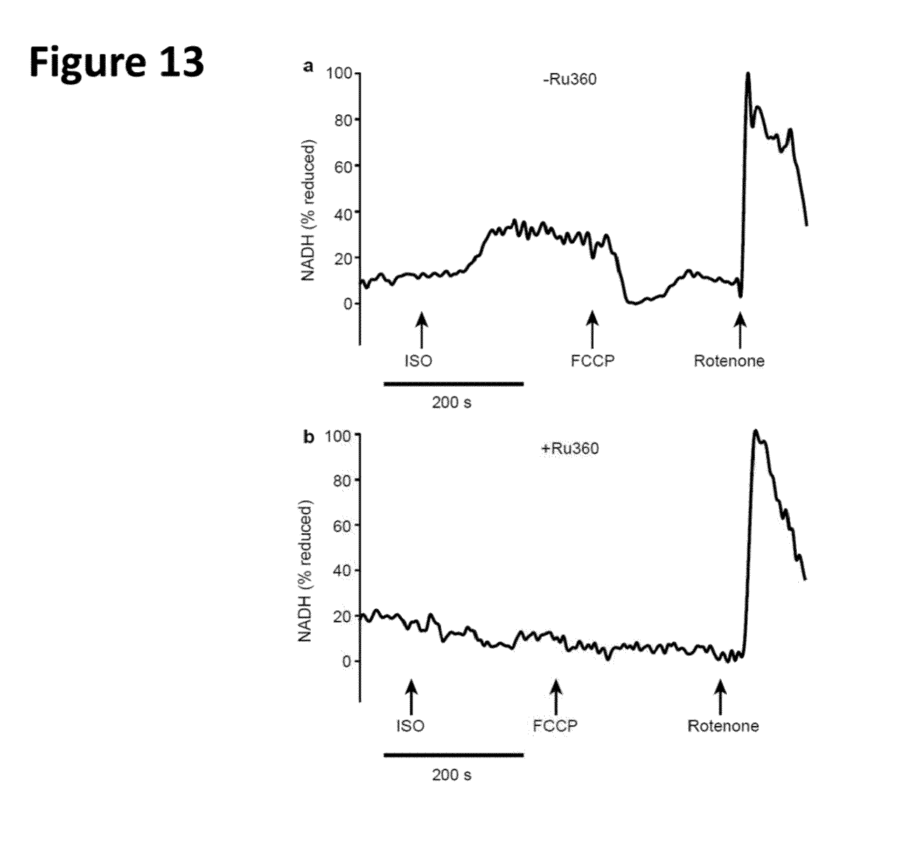

Mitochondrial Ca²⁺ entry increases oxidative phosphorylation by enhancing the activity of key mitochondrial dehydrogenases to provide NADH/NADPH reducing equivalents required for ATP synthesis.¹⁸ This mechanism is activated when cellular Ca²⁺ enters the inner mitochondrial membrane space from the cytosol through the MCU pathway.¹⁹ We next asked if mitochondrial Ca²⁺ entry was critical for oxidative phosphorylation-dependent ATP synthesis in SAN pacemaker cells. We infected cultured mouse SAN cells with adenovirus encoding mt-pericam²⁰, a circularly permutated Ca²⁺-sensitive fluorescent protein, to measure mitochondrial Ca²⁺ concentration ([Ca²⁺]ₘᵢₜₒ). The mt-pericam expression was localized to mitochondria in adenovirus infected SAN pacemaker cells, based on co-localization with MitoTracker Orange (FIG. 3a). ISO caused an increase in [Ca²⁺]ₘᵢₜₒ, (FIG. 3b) that was prevented by dialysis of Ru360 (FIG. 3b-d). Mitochondrial Ca²⁺ enhances ATP production by augmenting NADH, the primary electron donor for electron transport.²¹ We measured NADH fluorescence at baseline and after addition of ISO. ISO increased NADH fluorescence and this increase was prevented by Ru360 (FIG. 4a-b, FIG. 13), suggesting that ISO enhancement of NADH required MCU-mediated mitochondrial Ca²⁺ entry. These data confirm the MCU-dependence of coupling between [Ca²⁺]_{cyto} and [Ca²⁺]ₘᵢₜₒ in SAN cells and show that the MCU provides critical metabolic support for SERCA2a activity and SR Ca²⁺ loading. Together these data are consistent with a model where MCU-mediated enhancement of oxidative phosphorylation is a metabolic mechanism enabling the physiological fight or flight stress response in cardiac pacemaker cells (FIG. 4c).

Our data provide new mechanistic understanding into the fight or flight response to physiological stress by showing that heart rate increases rely on the MCU in cardiac pacemaker cells. Oxidative phosphorylation is enhanced by [Ca²⁺]ₘᵢₜₒ, which is required to generate ATP that fuels SERCA2a activity. Our findings show that Ca²⁺ homeostatic mechanisms in pacemaker cells form the framework for fight or flight heart rate increases but do not exclude additional modulation of heart rate by other Ca²⁺ sensitive signals^{22,23} or by Ca²⁺ independent ionic currents⁹. The MCU metabolic pathway appears optimized to generate heart rate increases during episodes of high energy demand that are signaled by catecholamines. Our data are consistent with earlier work showing mitochondrial Ca²⁺ is required to optimize refilling of intracellular Ca²⁺ stores by SERCA²⁴ and where ATP dialysis (3 mM) recovered Ca²⁺ sequestration by intracellular stores after addition of mitochondrial toxins.²⁵ Our findings provide insight into recent work showing MCU knock out selectively impairs high workload activity in striated muscle.²⁶ However, our data show that basal pacemaker cell activity is uncoupled from MCU-dependent ATP production. Because the relationship between MCU and SERCA2a in pacemaker cells appears purposed to selectively enable heart rate acceleration, future therapies targeting MCU or SERCA2a in pacemaker cells could provide a means to fine tune heart rates by preventing excessive heart rates without reducing resting heart rates.

Methods Summary

In vivo ECG was measured in DN-MCU and littermate WT mice using a computer-based data acquisition system (Powerlab 16/30; ADI instruments). Ex vivo heart rates were measured in isolated Langendorff-perfused hearts 7 days after SAN gene painting. The success of gene transfer to SAN in gene painted hearts was confirmed by immunostaining of HCN4 (enriched in SAN tissue) and GFP in SAN tissue sections (FIG. 8) obtained after experiments shown in (FIG. 1i-k), as described⁸. SAN expression of mt-pericam and DN-MCU was accomplished by viral infection of cultured SAN tissue. Single SAN cells were freshly isolated from SAN tissues from C57 mice or from cultured, virus infected SAN tissues. The success of gene transfer in cultured SAN tissue was confirmed by GFP fluorescence (see FIG. 3a). The isolated SAN cells were stored at 4° C. and studied within 7 hours of dissociation.

SAN cells were placed in Tyrode's solution at 36±0.5° C. and were identified by their characteristic morphology (spindle or spider shape) and spontaneous activity. SAN cells were identified electrophysiologically by spontaneous action potentials with a slow depolarizing phase 4 and the hyperpolarization-activated current (I_{f}).²⁷ Spontaneous action potentials, I_{f} and I_{Ca} were recorded using the perforated (amphotericin B or β-escin in studies requiring dialysis of Ru360 and/or ATP) patch-clamp technique²⁸ on single SAN cells at 36±0.5° C. in Tyrode's solution. Intracellular Ca²⁺ transients were recorded from single SAN cells using Fura-2 AM excited at 340 nm and 380 nm and emitted and 510 nm. Endogenous NADH from single SAN cells was measured by excitation at 350 nm and recording emitted light at 460 nm.²⁹ Mitochondrial Ca²⁺ was measured at 535 nm from mt-pericam expressing SAN cells exited at 415 nm²⁰. Mitochondrial Ca²⁺ entry was measured in cell membrane permeabilized cardiomyocytes isolated from DN-MCU transgenic, wild type mice and from HEK293 cells using Calcium green-5N³⁰.

Detailed Methods

All experiments were carried out in accordance with the guidelines of Institutional Animal Care and Use Committee (PHS Animal Welfare Assurance, A3021-01).

SAN Cell Isolation and Electrophysiological Recordings.

Isolation of single SAN cells from mice was performed according to previously published methods^{22, 27, 31} with minor modifications. Mice were administered an intraperitoneal injection of avertin (20 μl/g) and monitored until unresponsive. The heart was excised and placed into Tyrode's solution (35° C.), consisting of (mM) 140 NaCl, 5.0 HEPES, 5.5 Glucose, 5.4 KCl, 1.8 CaCl₂, and 1.0 MgCl₂. The pH was adjusted to 7.4 with NaOH. The SAN region, delimited by the crista terminalis, atrial septum and orifice of superior vena cava, was dissected free from the heart. The SAN was cut into smaller pieces, which were transferred and rinsed in a solution containing (mM) 140 NaCl, 5.0 HEPES, 5.5 Glucose, 5.4 KCl, 0.2 CaCl₂, 0.5 MgCl₂, 1.2 KH₂PO₄, 50 Taurine and 1 mg/ml bovine serum albumin (BSA), with pH adjusted to 7.4 using NaOH. SAN tissue pieces were digested in 5 ml of solution containing collagenase type I, elastase (Worthington), and protease type XIV (Sigma) for 20-30 min. The tissue was transferred to 10 ml of Kraft-Bruhe (KB) medium containing (mM) 100 potassium glutamate, 5.0 HEPES, 20 Glucose, 25 KCl, 10 potassium aspartate, 2.0 MgSO₄, 10 KH₂PO₄, 20 taurine, 5 creatine, 0.5 EGTA, and 1 mg/ml BSA, with pH adjusted to 7.2 using KOH. The tissue was agitated using a glass pipette for 10 min. The cells were stored at 4° C. and studied within 7 hours.

SAN cells were placed in Tyrode's solution at 36±0.5° C. SAN cells were identified by their characteristic morphology (spindle or spider shape) and spontaneous activity in all single cell experiments. SAN cells were also identified electrophysiologically by typical spontaneous action potentials with slow depolarizing phase 4 and the hyperpolarization-activated current (I_{f}) in electrophysiological experiments.

Spontaneous action potentials (APs) and I_{f} were recorded using the perforated (amphotericin B or β-escin) patch-clamp technique²⁸ on single SAN cell at 36±0.5° C. in Tyrode's solution (0.5 mM BaCl₂ was added to the bath solution when recording I_{f}³²). The pipette was filled with (mM) 130 potassium aspartate, 10 NaCl, 10 HEPES, 0.04 CaCl₂, amphotericin B 240 μg/ml with pH adjusted to 7.2 with KOH. SAN cells with stable APs lasting at least 5 min were included in the experiments. β-escin 25 μM was substituted for amphotericin B to allow dialysis of Ru360 or ATP. When recording I_{f}, membrane potential was held at −35 mV, the voltage steps were applied for 5 s ranging from −125 mV to −45 mV in 10 mV increments or vice versa.³³ I_{Ca} was measured using the perforated patch technique at 36±10.5° C. as previously described.²² I_{Ca} was confirmed by its sensitivity to Nifedipine 5 μM. Depolarizing voltage pulses (300 ms in duration) to various potentials (−60 mV to 60 mV in 10 mV step) were applied after 50 ms at −40 mV to inactivate I_{Na} from a holding potential of −70 mV. The pipette solution comprised (mM): 120 CsCl, 10 EGTA, 10 HEPES, 10 tetraethylammonium chloride (TEA), 5.0 phosphocreatine, 3.0 CaCl₂, 1.0 MgATP, 1.0 NaGTP and the pH was adjusted to 7.2 with 1.0 N CsOH. The bath (extracellular) solution comprised (mM): 137 NaCl, 10 HEPES, 10 glucose, 1.8 CaCl₂, 0.5 MgCl₂, 25 CsCl, pH was adjusted to 7.4 with NaOH.

Viral Infection with 5mt-Pericam/eGFP and DN-MCU.

Freshly isolated mouse SAN tissue was cut into small pieces and put into 35 mm tissue culture plates with DMEM. Fresh medium containing Ad-eGFP, Ad-5mt-pericam or Ad-DN-MCU was added to the plates at a multiplicity of infection of 100. Ad-ratio-5mt-pericam and Ad-DN-MCU were generated as follows: Ratiometric-Pericam-mt²⁰ was first subcloned into pacAd5CMV-mcs-KN (University of Iowa Gene Transfer Vector Core) using HindIII and EcoRI. As the localization of this Pericam with a single Cox4 targeting sequence was not exclusive to mitochondria, four additional mitochondrial targeting sequences were added to the amino terminus. Two tandem Cox8a targeting repeat units from the pcDNA3-D4cpv vector³⁴ were subcloned via Hind III into pacAd5CMV-ratiometric-Pericam-mt. Colonies were screened to identify clones that contained four tandem Cox8a inserts and confirmed via sequencing. For Ad-DN-MCU containing a C-terminal Myc tag, human MCU cDNA clone (GenBank: BC034235) was first obtained from the I.M.A.G.E consortium (ID: 5296557) and subcloned into pAd5CMVmcsIRESeGFP (University of Iowa Gene Transfer Vector Core, Iowa City, Iowa, U.S.A.) by PCR using Phusion DNA Polymerase (New England Biolabs) and the GeneArt Seamless Cloning and Assembly Kit (Life Technologies). PCR primers amplifying Myc-tagged MCU were: forward 5′-ATA AGC TTA TGG CGG CCG CCG CAG GTA GAT CG-3′ (SEQ ID NO:1), reverse 5′-CTA CAG GTC TTC TTC GCT AAT CAG TTT CTG TTC ATC TTT TTC ACC AAT TTG TCG GAG-3′ (SEQ ID NO:2), and pAd5CMVmcsIRESeGFP: forward 5′-GAA GAA GAC CTG TAG GAT ATC GAA TTC CTG CAG CCC-3′ (SEQ ID NO:3), reverse 5′-GCC GCC ATA AGC TTA TCG ATA CCG TCG ACC TC-3′ (SEQ ID NO:4). Dominant negative mutations in MCU that inhibit Ca²⁺ conductance (D260Q, E263Q)^{3,4} were generated with Agilent's QuikChange site-directed mutagenesis kit. Positive clones were confirmed by DNA sequencing. Adenoviruses expressing of 5mt-Pericam and DN-MCU-myc were generated by the University of Iowa Gene Transfer Vector Core. Expression of the recombinant ratio-5mt-pericam and DN-MCU in SAN cells was detected by GFP fluorescence. Recombinant adenovirus that expresses eGFP only (Ad-eGFP) was used as a control.

NADH and Mitochondrial Calcium Measurements.

The autofluorescence of endogenous NADH, which derives primarily from mitochondria,^{29,35} was measured as described²⁹. NADH was excited at 350 nm (AT350/SOX, Chroma) and fluorescence was recorded at 460 nm (ET460/50m and T400LP, Chroma). We normalized NADH level with FCCP as 0%, Rotenone induced NADH change as 100%. The baseline level of NADH was 22±3 of the rotenone-induced maximal value.

Mt-pericam, a mitochondrial matrix-targeted, circularly-permuted green fluorescent protein fused to calmodulin and its target peptide M13²⁰. Pericam emission at 535 nm due to excitation at 415 nm reports changes in Ca²⁺.³⁶ We used Mt-pericam to measure Ca²⁺ₘᵢₜₒ in isolated, cultured SAN cells. The mitochondrial Ca²⁺ level was monitored in cells transiently expressing the Mt-pericam protein at excitation wavelengths of 415 nm, presented as 1-F/F₀(Ca²⁺ₘᵢₜₒ)³⁶⁻³⁸, and the emission collected using a 535-nm band-pass filter.

Intracellular Ca²⁺ Transients.

Cytosolic Ca²⁺ levels were recorded from Fura-2-loaded cells, excited at wavelengths of 340 and 380 nm, and imaged with a 510 nm long-pass filter. Single isolated SAN cells were loaded with 0.1 μM Fura-2 AM for 20 minutes, and then perfused for 20 minutes to de-esterfy the Fura-2 AM in normal Tyrode's solution. After placement on a recording chamber, the cells were perfused in normal Tyrode's solution at 36° C.±0.5. Spindle-shaped, spontaneously beating cells were chosen for experiments. Action potential recording was performed simultaneously.

SAN Gene Painting.

SAN painting was performed as previously described.^{8,39} Briefly, Poloxamer 407 (Spectrum), trypsin (Sigma) and collagenase, type II (Worthington) mixture was made with 40% poloxamer, 1% trypsin and 0.25% collagenase in PBS, and then added to equal volume of recombinant adenovirus expressing plasmid for the gene(s) of interest (DN-MCU-IRES GFP vs eGFP) in solution. This mixture was liquid in consistency at 4° C., but gelled at 37° C. Mice were anesthetized using ketamine/xylazine (87.5/12.5 mg/kg respectively), intubated and ventilated. The junction of the superior vena cava and right atrium was visualized through a small incision in the 2nd intercostal space. The gel was applied to the posterior surface of the junction of the superior vena cava and right atrium with a fine brush. The intercostal muscles, pectoralis major and minor and the skin incision were closed using 6/0 silk and the mice were allowed to recover.

Ex Vivo Langendorff-Perfused Heart Rate Measurements.

ECG recording from Langendorff-perfused hearts was performed as described²². Briefly, excised hearts were quickly mounted on a modified Langendorff apparatus (HSE-HA perfusion systems, Harvard Apparatus, Holliston, Mass.) for retrograde aortic perfusion at a constant pressure of 80 mm Hg with oxygenated (95% O₂, 5% CO₂) Krebs-Henseleit solution consisting of (mM) 25 NaHCO₃, 118.5 NaCl, 4.0 KCl, 1.2 MgSO₄, 1.2 NaH₂PO₄, 1.5 CaCl₂, and 11.2 glucose, with pH equilibrated to 7.4. Each perfused heart was immersed in a water-jacketed bath and was maintained at 36° C. ECG measurements from the intact heart were continuously recorded with Ag⁺—AgCl electrodes, which were positioned around the heart in an approximate Einthoven configuration. After the heart was allowed to stabilize for 15 minutes, different concentrations of isoproterenol were added to the perfusate.

Immunofluorescence Staining.

Cryosections from the SAN area and control left atrium were stained as described⁸ using HCN4 (Abcam) and Myc epitope tag (Rockland) antibodies. Sections were imaged on an EVOS FL Auto microscope (Life Technologies).

Calcium Green Mitochondrial Ca²⁺ Uptake Assays.

We measured mitochondrial Ca²⁺ uptake using permeabilized HEK cells as previously described^{30,40}. Briefly, cells were grown in DMEM, with 10% FBS and 1% of Penicillin/Streptomycin. At 80% confluence cells were infected with DN-MCU adenovirus at MOI 10. Cells were harvested after 24 h incubation and placed into a 96 well plate. Each well was loaded with 1 million cells in respiratory buffer containing 125 mM KCl, 2 mM K₂HPO₄, 20 mM HEPES, 5 mM glutamate, 5 mM malate, 0.005% saponin, and 5 μM thapsigargin. 5 μM Ca²⁺ was injected at 3 minute intervals. Fluorescence was measured using a Tecan plate reader. Adult cardiac myocytes were isolated from 6-8 week old Wt or DN-MCU TG mice using a previously described isolation procedure.⁴⁰ 50 nM Blebbistatin was included in the myocyte buffer to prevent cellular contraction. Fluorescence intensity was measured from 50,000 cardiac myocytes/well. 100 μM Ca²⁺ was injected at 3 min intervals for myocyte experiments.

DN-MCU Overexpressing Mice.

The inter-membrane D₂₆₀IME₂₆₃ amino acid motif of human MCU was mutated to the dominant-negative (DN) form⁴, QIMQ, by replacing the nucleotide sequence, gacatcatggag with cagatcatgcag using site-directed mutagenesis (agilent.com). The resulting DN-MCU DNA product was amplified by PCR with Phusion DNA polymerase using a forward primer containing a SalI restriction site (5′-ACC AAC GTC GAC ATG GCG GCC GCC GCA GGT AG-3′ (SEQ ID NO:5)) and reverse primer containing a C-terminal myc epitope tag sequence and HindIII restriction site (5′-GAA CGC AAG CTT CCT ACA GGT CTT CTT CGC TAA TCA GTT TCT GTT CAT CTT TTT CAC CAA TTT GTC GGA G-3′ (SEQ ID NO:6)). PCR products were digested and ligated into SalI and HindIII digested pBS-αMHC-script-hGH vector and positive clones confirmed by sequencing. Mouse embryonic stem cells were injected with the linearized DNA (digested with NotI) in the University of Iowa Transgenic Mouse Core Facility and implanted into pseudo-pregnant females to generate B6XSJL F1 mice. Insertion of the transgene into the mouse genome was confirmed by PCR analysis (not shown) using the forward primer, CCCACACCAGAAATGACAGACAGAT (SEQ ID NO:7) and reverse primer, AGAGGAGCAGCAGGAGCGATCTA (SEQ ID NO:8), producing a product of 200 bases. Mice were backcrossed to F4 generation or greater into the CD1 background. Transgenic and control mice of either gender were sacrificed at the age of 2-3 months.

Western Blots for Detecting Phospholamban.

Heart lysates were prepared from flash-frozen mouse right atria and western blotting was performed with a SDS-PAGE electrophoresis system as described previously²². Briefly, 30 μg protein samples were size-fractionated on SDS-PAGE, and then transferred to PVDF membranes. The membranes were probed with anti-pSer16-PLN (1:5,000), anti-pThr17-PLN (1:5,000) (from Badrilla Ltd., Leeds, United Kingdom) at room temperature for 4 hours. Then membranes were incubated with Alexa-Fluor680-conjugated anti-mouse (Invitrogen Molecular Probes, Carlsbad, Calif.) and/or IR800Dyeconjugated anti-rabbit fluorescent secondary antibodies (Rockland Immunochemicals, Gilbertsville, Pa.), and scanned on an Odyssey infrared scanner (Li-Cor, Lincoln, Nebr.). Integrated densities of protein bands were measured using ImageJ Data Acquisition Software (National Institute of Health, Bethesda, Md.).

Western Blots for Detecting Myc Tagged DN-MCU.

Heart, liver and skeletal muscle tissues were harvested and flash frozen. Samples were homogenized in RIPA buffer (10 mM Tris-Cl, pH8.0, 1 mM EDTA, 1% Triton X-100, 0.1% sodium deoxycholate, 0.1% SDS, 140 mM NaCl), containing protease and phosphatase inhibitors with antioxidant. 20 μg of protein were fractionated on NuPAGE (Invitrogen) 4-12% SDS-PAGE gels and transferred overnight to PVDF membranes (Bio-Rad). Nonspecific binding was blocked with 10% w/v non-fat milk powder in TBS-T (50 mM Tris-HCl, pH 7.6, 150 mM NaCl, 27 mM KCl and 0.25% Tween-20). The membranes were probed with anti-MYC (1:1000) (Rockland Immunochemicals, Gilbertsville, Pa.) at room temperature for 2 hours. Then membranes were incubated with anti-rabbit HRP-conjugated secondary antibody. Protein bands were visualized using ECL reagent (Lumi-Light, Roche). Correct loading was confirmed with Coomassie staining of membranes.

Mouse Surface Electrocardiograms.

Mouse surface electrocardiogram (ECG) tracings were acquired as described.⁴¹ Prior to the ECG acquisition mice were pre-anesthetized with 2% isoflurane in II oxygen/min (Isotec100 Series, Isoflurane Vaporizer, Harvard Apparatus, Holliston, USA). Mice were placed in a supine position on a heated ECG pad (Mousepad, THM 100, Indus Instruments, Webster, USA), and the limbs were attached to the pad electrodes using a tape to obtain ECG lead II. Anesthesia was maintained via facemask by continuous isoflurane ventilation as described above. The body temperature was continuously monitored using a rectal probe and sustained within 36-37° C. To obtain a baseline ECG, animals were allowed to rest for 5 min after being positioned on the pad. Thereafter Isoproterenol (10 μg/kg) was injected i.p. ECG acquisition was performed continuously using a multichannel amplifier and data acquisition system (Powerlab 16/30, AD Instruments, Colorado Springs, USA) converting the signal into digital for a further data analysis (Labchart Pro software, version 7, AD Instruments, Colorado Springs, USA).

Statistical Analysis.

Data are presented as mean±SEM, unless otherwise noted. Statistical analysis was performed either with 1-way ANOVA or an unpaired or paired Student's t test, as appropriate. Analyses were performed with Sigmaplot or Sigmastat (Systat Software, Inc. San Jose, Calif. 95110 USA). The null hypothesis was rejected for a P<0.05.

### REFERENCES

1. Cannon, W. B. The Wisdom of the Body. New York, N.Y., US: W W Norton & Co. 312pp (1932)
2. Bers, D. M. Excitation-Contraction Coupling and Cardiac Contractile Force. 2nd ed. Dordrecht, Netherlands: Kluwer Academic Press. 427pp (2001)
3. De Stefani, D., Raffaello, A., Teardo, E., Szabo, I. & Rizzuto, R. A forty-kilodalton protein of the inner membrane is the mitochondrial calcium uniporter. Nature 476, 336-340 (2011)
4. Baughman, J. M. et al. Integrative genomics identifies MCU as an essential component of the mitochondrial calcium uniporter. Nature 476, 341-345 (2011)
5. Glancy, B. & Balaban, R. S. Role of mitochondrial Ca2+ in the regulation of cellular energetics. Biochemistry 51, 2959-73 (2012)
6. Irisawa, H., Brown, H. F. & Giles W. Cardiac pacemaking in the sinoatrial node. Physiol. Rev. 73, 197-227 (1993)
7. Gupta, A., Chacko, V. P. & Weiss, R. G. Abnormal energetics and ATP depletion in pressure-overload mouse hearts: in vivo high-energy phosphate concentration measures by noninvasive magnetic resonance. Am. J. Physiol. Heart Circ. Physiol. 297, H59-64 (2009)
8. Swaminathan, P. D. et al. Oxidized CaMKII causes cardiac sinus node dysfunction in mice. J. Clin. Invest. 121, 3277-88 (2011)
9. Mangoni, M. E. & Nargeot, J. Genesis and regulation of the heart automaticity. Physiol. Rev. 88, 919-82 (2008)
10. Sanders, L., Rakovic, S., Lowe, M., Mattick, P. A. & Terrar, D. A. Fundamental importance of Na+-Ca2+ exchange for the pacemaking mechanism in guinea-pig sino-atrial node. J. Physiol. 571, 639-49 (2006)
11. Rigg, L., Heath, B. M., Cui, Y. & Terrar, D. A. Localization and functional significance of ryanodine receptors during β-adrenergic stimulation in guinea-pig sino-atrial node. Cardiovasc. Res. 48, 254-264 (2000)
12. Verjovski-Almeida, S. & Inesi, G. Fast-kinetic evidence for an activating effect of ATP on the Ca2+ transport of sarcoplasmic reticulum ATPase. J. Biol. Chem. 254, 18-21(1979)
13. Cavagna, M., O'Donnell, J. M., Sumbilla, C., Inesi, G. & Klein, M. G. Exogenous Ca2+-ATPase isoform effects on Ca2+ transients of embryonic chicken and neonatal rat cardiac myocytes. J. Physiol. 528, 53-63 (2000)
14. Zhai, J., et al. Cardiac-specific overexpression of a superinhibitory pentameric phospholamban mutant enhances inhibition of cardiac function in vivo. J. Biol. Chem. 275, 10538-44 (2000)
15. Simmerman, H. K. & Jones, L. R. Phospholamban: protein structure, mechanism of action, and role in cardiac function. Physiol Rev. 78, 921-47 (1998)
16. Liao, Z., Lockhead, D., Larson, E. D. & Proenza, C. Phosphorylation and modulation of hyperpolarization-activated HCN4 channels by protein kinase A in the mouse sinoatrial node. J. Gen. Physiol. 136, 247-258 (2010)
17. Mangoni, M. E., et al. Voltage-dependent calcium channels and cardiac pacemaker activity: from ionic currents to genes. Prog. Biophys. Mol. Biol. 90, 38-63 (2006)
18. McCormack, J. G., Halestrap, A. P. & Denton, R. M. Role of calcium ions in regulation of mammalian intramitochondrial metabolism. Physiol. Rev. 70, 391-425 (1990)
19. Rizzuto, R., De Stefani, D., Raffaello, A. & Mammucari, C. Mitochondria as sensors and regulators of calcium signalling. Nat. Rev. Mol. Cell Biol. 13, 566-78 (2012)
20. Nagai, T., Sawano, A., Park, E. S. & Miyawaki. A. Circularly permuted green fluorescent proteins engineered to sense Ca²⁺. Proc. Natl. Acad. Sci. USA. 98, 3197-202 (2001)
21. Territo, P. R., Mootha, V. K., French, S. A. & Balaban, R. S. Ca(2+) activation of heart mitochondrial oxidative phosphorylation: role of the F(0)/F(1)-ATPase. Am. J. Physiol. Cell Physiol. 278, C423-35 (2000)
22. Wu, Y., et al. Calmodulin kinase II is required for fight or flight sinoatrial node physiology. Proc. Natl. Acad. Sci. USA. 106, 5972-5977 (2009)
23. Vinogradova, T. M., Bogdanov, K. Y. & Lakatta, E. G. beta-Adrenergic stimulation modulates ryanodine receptor Ca(2+) release during diastolic depolarization to accelerate pacemaker activity in rabbit sinoatrial nodal cells. Circ. Res. 90, 73-9 (2002)
24. Landolfi, B., Curci, S., Debellis, L., Pozzan, T. & Hofer, A. M. Ca2+ homeostasis in the agonist-sensitive internal store: functional interactions between mitochondria and the ER measured In situ in intact cells. J. Cell Biol. 142, 1235-43 (1998)
25. Hofer, A. M., Schlue, W. R., Curci, S. & Machen, T. E. Spatial distribution and quantitation of free luminal [Ca] within the InsP3-sensitive internal store of individual BHK-21 cells: ion dependence of InsP3-induced Ca release and reloading. FASEB J. 9, 788-98 (1995)
26. Pan, X., et al. The physiological role of mitochondrial calcium revealed by mice lacking the mitochondrial calcium uniporter. Nat. Cell Biol. 15, 1464-72 (2013)
27. Mangoni, M. E. & Nargeot, J. Properties of the hyperpolarization-activated current (I(f)) in isolated mouse sino-atrial cells. Cardiovasc. Res. 52, 51-64 (2001)
28. Rae, J., Cooper, K., Gates, P. & Watsky, M. Low access resistance perforated patch recordings using amphotericin B. J. of Neuroscience Methods 37, 15-26 (1991)
29. Eng, J., Lynch, R. M. & Balaban, R. S. Nicotinamide adenine dinucleotide fluorescence spectroscopy and imaging of isolated cardiac myocytes. Biophys. J. 55, 621-30 (1989)
30. Murphy, A. N., Bredesen, D. E., Cortopassi, G., Wang, E. & Fiskum, G. Bcl-2 potentiates the maximal calcium uptake capacity of neural cell mitochondria. Proc. Natl. Acad. Sci. USA. 93, 9893-8 (1996
31. DiFrancesco, D., Ferroni, A., Mazzanti, M. & Tromba, C. Properties of the hyperpolarizing-activated current (if) in cells isolated from the rabbit sino-atrial node. J. Physiol. 377, 61-88 (1986)
32. Cho, H. S., Takano, M. & Noma, A. The electrophysiological properties of spontaneously beating pacemaker cells isolated from mouse sinoatrial node. J. Physiol. 550, 169-180 (2003)
33. Baruscotti, M., et al. Deep bradycardia and heart block caused by inducible cardiac-specific knockout of the pacemaker channel gene Hcn4. Proc. Natl. Acad. Sci. USA. 108, 1705-10 (2011)
34. Palmer, A. E., et al. Ca2+ indicators based on computationally redesigned calmodulin-peptide pairs. Chem. Biol. 13, 521-30 (2006)
35. Aon, M. A., Cortassa, S., Marban, E. & O'Rourke, B. Synchronized whole cell oscillations in mitochondrial metabolism triggered by a local release of reactive oxygen species in cardiac myocytes. J. Biol. Chem. 278, 44735-44744 (2003
36. Jiang, D., Zhao, L. & Clapham, D. E. Genome-wide RNAi screen identifies Letm1 as a mitochondrial Ca2+/H+ antiporter. Science 326(5949), 144-7 (2009)
37. Feng, S., et al. Canonical transient receptor potential 3 channels regulate mitochondrial calcium uptake. Proc. Natl. Acad. Sci. USA. 110, 11011-6 (2013)
38. Palty, R., et al. NCLX is an essential component of mitochondrial Na+/Ca2+ exchange. Proc. Natl. Acad. Sci. USA. 107, 436-41 (2010)
39. Kikuchi, K., McDonald, A. D., Sasano, T. & Donahue, J. K. Targeted modification of atrial electrophysiology by homogeneous transmural atrial gene transfer. Circulation 111, 264-70 (2005)
40. Joiner, M. L., et al. CaMKII determines mitochondrial stress responses in heart. Nature 491(7423):269-73 (2012)
41. Purohit, A., et al. Oxidized Ca(2+)/calmodulin-dependent protein kinase II triggers atrial fibrillation. Circulation 128, 1748-57 (2013)

### Example 2

Reference is made to Rasmussen et al., “Inhibition of MCU forces extramitochondrial adaptations governing physiological and pathological stress responses in heart,” Proc. Natl. Acad. Sci USA 2015 July 21; 112(29):9129-34, the content of which is incorporate herein by reference in its entirety.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method of treating or preventing a disease or disorder associated with mitochondrial calcium channel (MCU) activity in a patient in need thereof, the method comprising administering a pharmaceutical composition comprising an agent that modulates MCU activity to the patient.

2. The method of claim 1, wherein the disease or disorder is cardiac arrhythmia.

3. The method of claim 2, wherein the arrhythmia is atrial fibrillation.

4. The method of claim 1, wherein the method reduces heart rate acceleration in the patient.

5. The method of claim 1, wherein the method does not reduce basal heart rate in the patient.

6. The method of claim 1, wherein the method reduces heart rate acceleration in the patient by at least about 50%.

7. The method of claim 1, wherein the method does not reduce basal heart rate in the patient by more than 10%.

8. The method of claim 1, wherein the agent that modulates MCU activity is an MCU inhibitor.

9. The method of claim 8, wherein the MCU inhibitor is an MCU antagonist.

10. The method of claim 9, wherein the MCU antagonist is Ru360.

11. The method of claim 8, wherein the MCU inhibitor is a polynucleotide encoding a dominant negative mutant of MCU.

12. The method of claim 11, wherein the polynucleotide is expressed from a recombinant viral expression vector.

13. The method of claim 11, wherein the polynucleotide is administered via gene painting on cardiac tissue.

14. A pharmaceutical composition comprising a polynucleotide encoding a dominant negative mutant of MCU.

15. The pharmaceutical composition of claim 14, wherein the polynucleotide is present in a viral vector.

16. The pharmaceutical composition of claim 15, wherein the viral vector is an adenovirus vector.
